(19) United States
(12) Patent Application Publication
(10) Pub. No.: US 2020/0283858 A1
(43) Pub. Date: Sep. 10, 2020

(54) MEASUREMENT OF ENDOGENOUS RETROVIRUS EXPRESSION TO GUIDE IMMUNOTHERAPY IN CANCER

(71) Applicants: Rutgers, The State University of New Jersey, New Brunswick, NJ (US); Vanderbilt University, Nashville, TN (US)

(72) Inventors: Shridar Ganesan, New Brunswick, NJ (US); Gyan Bhanot, Piscataway, NJ (US); Anshuman Panda, New Brunswick, NJ (US); W. Kimryn Rathmell, Nashville, TN (US); Aguirre Andres de Cubas, Nashville, TN (US)

(73) Assignees: Rutgers, The State University of New Jersey, New Brunswick, NJ (US); Vanderbilt University, Nashville, TN (US)

(21) Appl. No.: 16/812,204

(22) Filed: Mar. 6, 2020

Related U.S. Application Data
(60) Provisional application No. 62/814,660, filed on Mar. 6, 2019.

(51) Int. Cl. C12Q 1/70; C12Q 1/6886

(52) U.S. Cl. CPC C12Q 1/70; C12Q 2600/158; C12Q 1/6886

(57) ABSTRACT

Methods of detecting immunogenic endogenous retroviruses and treating cancer are disclosed herein. In some embodiments, the methods include detecting increased expression of endogenous retroviruses in a tumor sample.

### CROSS REFERENCE TO RELATED APPLICATION

This application claims the benefit of the earlier filing date of U.S. Provisional Application No. 62/814,660, filed Mar. 6, 2019, which is incorporated by reference in its entirety.

### ACKNOWLEDGMENT OF GOVERNMENT SUPPORT

This invention was made with government support under R01-CA198482, K24-CA172355, T32-CA009582-28, and P30-CA072720, awarded by the National Institute of Health; CA160728P1, awarded by the Department of Defense; and PHY-1607611, awarded by the National Science Foundation.

### FIELD

This application provides methods of detecting immunogenic endogenous retrovirus expression and/or treating cancer.

### BACKGROUND

Immune checkpoint blockade (ICB) leads to durable objective responses in several cancer types (Kim and Eder, Oncology (Williston Park, 28 Suppl 3:15-28, 2014). A high mutation burden, such as from exposure to exogenous carcinogens (Snyder et al., N Engl J Med., 371(23):2189-2199, 2014; Rizvi et al., Science, 348(6230):124-8, 2015) or intrinsic defects in DNA repair and replication (Le et al., N Engl J Med., 372(26):2509-20, 2015) (Mehnert et al., J Clin Invest., 126(6):2334-40, 2016), predicts response to ICB in some cancer types (Panda et al., JCO Precis Oncol., 2017). Further, expression of certain exogenous viruses in tumors, such as Epstein-Barr virus in gastric cancer (Panda et al., J Natl Cancer Inst., 110(3):316-320, 2018) and NK/T-cell lymphoma (Kwong et al., Blood, 129(17):2437-2442, 2017), and Merkel-cell polyomavirus in Merkel-cell cancer (Nghiem et al., N Engl J Med., 374(26):2542-52, 2016; Kaufman et al., Lancet Oncol., 17(10):1374-1385, 2016), is also associated with response to ICB. However, some cancers, such as clear cell renal cell carcinoma (ccRCC), have clinically significant and durable responses to ICB (Motzer et al., N Engl J Med., 373(19):1803-13, 2015), despite low mutation burden and absence of known exogenous viral infection.

Loss of the chromatin modifying gene PBRM1 correlates with response to ICB in pre-treated ccRCC patients (Miao et al., Science, 359(6377):801-806, 2018). Further, in multiple cohorts of ccRCC patients, tumors with PBRM1 loss have lower levels of the CD8+ T-cell marker (CD8A), interferon gamma (IFNG), and immune checkpoint genes compared to tumors with intact PBRM1 (Miao et al., Science, 359(6377):801-806, 2018); however, the underlying mechanism is unknown. Moreover, ccRCC, while having low overall mutation burden, are enriched in frameshift mutations and may be more immunogenic (Turajlic et al., Lancet Oncol., 18(8):1009-1021, 2017). However, the relationship between levels of frameshift mutations and response to ICB remains unclear.

### SUMMARY

Disclosed herein are methods of detecting immunogenic endogenous retroviruses (πERVs) in a subject, including measuring expression of at least one endogenous retrovirus (ERV) in a solid tumor sample from a subject with a solid tumor; and measuring expression of at least one control sample from at least one of a tumor sample from a subject that is not responsive to at least one antagonist of an immune checkpoint pathway (ICP)-related molecule; and/or a sample from a subject without a tumor, wherein the at least one ERV is an πERV if the ERV expression is at least 1.5 fold, at least 2-fold, at least 3-fold, or at least 4-fold greater than the control sample. In some examples, if at least one πERV in the tumor is detected, the methods further include administering a therapeutically effective amount of a PD-1 antagonist, PD-L1 antagonist, CTLA4 antagonist, BTLA antagonist, HVEM antagonist, LAG-3 antagonist, or combinations thereof, to the subject having the tumor, thereby treating the tumor. In some examples, expression of the πERV is correlated with upregulation of at least one ICP. In some examples, the tumor sample from the subject responsive to at least one antagonist of an ICP-related molecule and/or the tumor sample from the subject that is not responsive to at least one antagonist of an ICP-related molecule is a solid tumor. In some examples, the solid tumor is a clear cell renal cell carcinoma, ER+ HER2− breast cancer, colon cancer, or head and neck squamous cell cancer. In some examples, the at least one ERV includes one or more of ERVK.3, ERV3-2, ERVK7, EVER24, or ERVK.8. In some examples, the at least one ERV includes or is ERV3-2. In some examples, the PD-1 antagonist, PD-L1 antagonist, CTLA4 antagonist, BTLA antagonist, HVEM antagonist, OR LAG-3 antagonist include a monoclonal antibody. In some examples, the PD-1 or PD-L1 antagonist includes one or more of tezolizumab, MPDL3280A, BNS-936558 (Nivolumab), pembrolizumab, pidilizumab, CT011, AMP-224, AMP-514, MEDI-0680, BMS-936559, BMS935559, MEDI-4736, MPDL-3280A, MSB-0010718C, MGA-271, indoximod, epacadostat, BMS-986016, MEDI-4736, MEDI-4737, MK-4166, BMS-663513, PF-05082566 (PF-2566), lirilumab, and durvalumab. In some examples, the CTLA4 antagonist includes or is tremelimumab, ipilimumab, or both.

In some examples, measuring expression of at least one ERV includes measuring ERV nucleic acid expression, such as through amplification of ERV nucleic acid molecules, for example, using the ΔΔCt method. In some examples, the amplification (such as using the ΔΔCt method) includes normalizing the ERV nucleic acid expression to nucleic acid expression of a housekeeping gene (such as hypoxanthine-guanine phosphoribosyltransferase (HPRT1)). For example, measuring ERV nucleic acid expression can include using (a) 5′-CAAGAGGCGGCATAGAAGCAA-3′ (SEQ ID NO: 1) and 5′-GGAGAGTAGCTTGGGGTTTCA-3′ (SEQ ID NO: 2); (b) 5′-AGCCATTTACAAAGAAAGGGGAC-3′ (SEQ ID NO: 3) and 5′-CTATGCCGCCTCTTGTCTGAT-3′ (SEQ ID NO: 4); or (c) both (a) and (b). In some examples, HPRT1 expression is used for normalization of ERV expression such as through using 5′-GACACTGGCAAAACAATGCAGAC-3′ (SEQ ID NO: 5); and 5′-TGGCTTATATCCAACACTTCGTGG-3′ (SEQ ID NO: 6).

Further disclosed herein are methods of treating cancer, including selecting a subject with cancer; detecting at least one immunogenic endogenous retrovirus (πERV) in the subject, including measuring expression of at least one endogenous retrovirus (ERV) in a tumor sample from the subject, wherein at least one ERV is an πERV if the ERV expression is at least 1.5 fold, at least 2-fold, at least 3-fold, or at least 4-fold greater than a control sample, wherein the control sample includes expression of the at least one ERV expected for at least one of: a tumor sample from a subject with cancer that is not responsive to at least one antagonist of an ICP-related molecule; and/or a sample from a subject without a tumor. In some examples, the methods include administering a therapeutically effective amount of a PD-1 antagonist, PD-L1 antagonist, CTLA4 antagonist, BTLA antagonist, HVEM antagonist, LAG-3 antagonist, or combinations thereof, thereby treating the cancer. In some examples, measuring expression of at least one ERV includes measuring ERV3-2 nucleic acid expression.

Also disclosed herein are methods of detecting immunogenic endogenous retroviruses (πERVs), including measuring expression of at least one endogenous retrovirus (ERV) in a tumor sample from a subject responsive to at least one antagonist of an immune checkpoint pathway (ICP)-related molecule; and measuring expression of at least one control sample from at least one of: a tumor sample from a subject that is not responsive to at least one antagonist of an immune checkpoint pathway (ICP)-related molecule; and/or a sample from a subject without a tumor.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1E: Potentially immunogenic ERVs (πERVs) are abundant in four solid cancers from The Cancer Genome Atlas (TCGA). [FIG. 1A] Immune checkpoint activation criteria used to identify potentially immunogenic ERVs (πERVs) in each solid cancer type. [FIG. 1B] Number of πERVs in each solid cancer type identified four cancer types with unusually high number of πERVs. [FIG. 1C] Correlation (Spearman) between expression of πERVs (rows) and levels of immunological variables (columns) in the four cancer types. [FIG. 1D-1E] ERV3-2 was identified as a πERV in 11 different cancer types. The cancer type acronyms are standard TCGA abbreviations (tcga-data.nci.nih.gov).

FIGS. 2A-2F: Expression of πERVs define subtypes with differential immune checkpoint activation in ccRCC (KIRC). [FIG. 2A] Hierarchical clustering of tumors from TCGA (columns) by expression (percentile) of πERVs (rows) stratifies tumors into three subtypes (H/I/L). [FIG. 2B] Frequency of VHL, PBRM1, SETD2, and BAP1 mutation (dark=truncating mutations, light=other non-synonymous mutations) in the three subtypes. Comparison of [FIGS. 2C-2D] overall immune infiltration in tumor (“ImmuneScore”) and fractional composition of tumor infiltrating leukocytes, and [FIGS. 2E-2F] mRNA expression of CD8A (cytotoxic T-cell marker) and immune checkpoint genes between πERV-high and πERV-low subtypes. Number of samples: [FIGS. 2C-2D] ImmuneScore (119 H, 228 L), rest (90 H, 134 L), [FIGS. 2E-2F] 119 H and 228 L. The p-values reported in bar plots and boxplots are from Fisher's exact test and Wilcoxon rank-sum test respectively (all two-sided).

FIGS. 3A-3C: RNA expression of ERV3-2 predicts response to immune checkpoint blockade in ccRCC. [FIG. 3A] Expression of ERV3-2 is significantly higher in tumors from responders compared with tumors from non-responders and is an excellent predictor of response to immune checkpoint blockade for both primers. Green arrows mark the optimal cutoffs that were subsequently used to stratify patients into ERV3-2+ or ERV3-2− group for consistency check. [FIG. 3B] ERV3-2+ group has significantly higher objective response rates and longer progression-free survival compared with ERV3-2− group for both primers. [FIG. 3C] In contrast, πERV-high/intermediate subtypes have shorter overall survival under standard therapy compared with πERV-low subtype in TCGA ccRCC (KIRC) cohort. Number of samples is specified in each panel. The p-values reported in bar plots, boxplots, and Kaplan-Meier plots are from Fisher's exact test, Wilcoxon rank-sum test, and log-rank tests respectively (all two-sided).

FIG. 4: Patient characteristics and ERV3-2 levels (for two different primers) in the validation cohort.

FIGS. 5A-5B: Top terms from enrichment analysis of genes whose expression levels follow the trends πERV-high>πERV-intermediate>πERV-low>adjacent normal or πERV-high<πERV-intermediate<πERV-low<adjacent normal in ccRCC, where > or < means significantly higher or lower respectively.

FIG. 6: Top gene ontology terms from enrichment analysis of genes whose expression levels correlate with overall ERV expression in ccRCC, ER+/HER2− breast cancer, and colon cancer.

FIGS. 7A-7E: Expression of πERVs define subtypes with differential immune checkpoint activation in ER+/HER2− breast cancer. [FIG. 7A] Hierarchical clustering of tumors (columns) by expression (percentile) of πERVs (rows) stratifies tumors into three subtypes (H/I/L). Comparison of [FIG. 7B] overall immune infiltration in tumor (“ImmuneScore”) and [FIG. 7C] fractional composition of tumor infiltrating leukocytes, and [FIG. 7D] mRNA expression of CD8A (cytotoxic T-cell marker) and immune checkpoint genes between πERV-high and πERV-low subtypes. [FIG. 7E] Percent of tumors with APOBEC mutagenesis in the three subtypes. Number of samples: [FIGS. 7B and 7D] 226 H and 185 L, [FIG. 7C] 152 H and 123 L. P-values reported in bar plots and boxplots are from Fisher's exact test and Wilcoxon rank-sum test respectively (all two-sided).

FIGS. 8A-8E: Expression of πERVs define subtypes with differential immune checkpoint activation in colon adenocarcinoma. [FIG. 8A] Hierarchical clustering of tumors (columns) by expression (percentile) of πERVs (rows) stratifies tumors into three subtypes (H/I/L). Comparison of [FIG. 8B] overall immune infiltration in tumor (“ImmuneScore”) and [FIG. 8C] fractional composition of tumor infiltrating leukocytes and percentages of regulatory T-cells among all T-cells, and [FIG. 8D] mRNA expression of CD8A (cytotoxic T-cell marker) and immune checkpoint genes between πERV-high and πERV-low subtypes. [FIG. 8E] Frequency of MSI-H tumors in the three subtypes. Number of samples: [FIGS. 8B and 8D] 128 H and 97 L, [FIG. 8C] 68 H and 29 L. P-values reported in bar plots and boxplots are from Fisher's exact test and Wilcoxon rank-sum test respectively (all two-sided).

FIGS. 9A-9E: Expression of πERVs define subtypes with differential immune checkpoint activation in head-neck squamous-cell carcinoma. [FIG. 9A] Hierarchical clustering of tumors (columns) by expression (percentile) of πERVs (rows) stratifies tumors into two subtypes (H/L). Comparison of [FIG. 9B] overall immune infiltration in tumor (“ImmuneScore”) and [FIG. 9C] fractional composition of tumor infiltrating leukocytes, and [FIG. 9D] mRNA expression of CD8A (cytotoxic T-cell marker) and immune checkpoint genes between πERV-high and πERV-low subtypes. [FIG. 9E] Frequency of HPV+ tumors in the two subtypes. Number of samples: [FIGS. 9B and 9D] 131 H and 170 L, [FIG. 9C] 119 H and 143 L. P-values reported in bar plots and boxplots are from Fisher's exact test and Wilcoxon rank-sum test respectively (all two-sided).

FIGS. 10A-10C: πERV expression is an independent predictor of immune checkpoint activation. πERV-high and πERV-low subtypes have differential immune checkpoint activation independently of [FIG. 10A] APOBEC enrichment status in ER+/HER2− breast cancer, [FIG. 10B] MSI-H status in colon cancer, and [FIG. 10C] HPV status in head-neck squamous-cell cancer. Number of samples: [FIG. 10A] −(165 H, 155 L), +(58 H, 25 L); [FIG. 10B] −(89 H, 84 L), +(39 H, 13 L); [FIG. 10C] −(91 H, 144 L), +(38 H, 26 L). P-values are from two-sided Wilcoxon rank-sum test.

FIGS. 11A-11C: Example ERVs of use in the methods described herein. (See, e.g., Mayer et al., “A revised nomenclature for transcribed human endogenous retroviral loci,” Mobile DNA, 2:7, 2011, incorporated herein by reference in its entirety).

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file, created on Mar. 6, 2020, 5 KB, which is incorporated by reference herein. In the accompanying sequence listing:

SEQ ID NOS: 1-4 are exemplary ERV3-2 primers.

SEQ ID NOS: 5-6 are exemplary HPRT1 primers.

SEQ ID NO: 7 is an exemplary ERV3-2 nucleic acid sequence.

SEQ ID NO: 8 is an exemplary ERV3-2 amino acid sequence.

### DETAILED DESCRIPTION

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising an ERV” includes single or plural ERVs and is considered equivalent to the phrase “comprising at least one ERV.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. Dates of GenBank® Accession Nos. referred to herein are the sequences available at least as early as Mar. 6, 2020. All references and GenBank® Accession numbers cited herein are incorporated by reference in their entirety.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

In order to facilitate review of the various embodiments of the disclosure, the following explanations of specific terms are provided:

Administration: To provide or give a subject an agent, such as an anti-cancer agent (e.g., antagonist of PD-1, PD-L1 and/or CTLA4), by any effective route. Exemplary routes of administration include, but are not limited to, injection (such as subcutaneous, subdermal, intramuscular, intradermal, intraperitoneal, intratumoral, and intravenous), transdermal, intranasal, oral, vaginal, rectal, and inhalation routes.

Cancer: A malignant tumor characterized by abnormal or uncontrolled cell growth. Other features often associated with cancer include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. “Metastatic disease” refers to cancer cells that have left the original tumor site and migrate to other parts of the body for example via the bloodstream or lymph system.

Contact: Placement in direct physical association, including a solid or a liquid form. Contacting can occur in vitro or ex vivo, for example, by adding a reagent to a sample (such as one containing tumor cells), or in vivo by administering to a subject.

Effective amount (or therapeutically effective amount): The amount of an agent (such as an anti-cancer therapy, such as an antagonist of PD-1, PD-L1 and/or CTLA4) that is sufficient to effect beneficial or desired results.

An effective amount (also referred to as a therapeutically effective amount) may vary depending upon one or more of: the subject and disease condition being treated, the weight and age of the subject, the severity of the disease condition, the manner of administration and the like, which can readily be determined by one of ordinary skill in the art. The beneficial therapeutic effect can include enablement of diagnostic determinations; amelioration of a disease, symptom, disorder, or pathological condition; reducing or preventing the onset of a disease, symptom, disorder or condition; and generally counteracting a disease, symptom, disorder or pathological condition.

In one embodiment, an “effective amount” (e.g., of an anti-cancer agent, such as an antagonist of PD-1, PD-L1 and/or CTLA4) may be an amount sufficient to reduce the volume/size of a tumor, the weight of a tumor, the number/extent of metastases, reduce the volume/size of a metastasis, the weight of a metastasis, or combinations thereof, for example by at least about 10%, at least about 20%, at least about 25%, at least about 50%, at least about 70%, at least about 75%, at least about 80%, at least about 90%, at least about 95%, or at least about 99% (for example as compared to no administration of the therapeutic agent, or as compared to administration of the same agent, but in a subject having a tumor with normal or decreased expression of one or more ERVs provided herein). In one embodiment, an “effective amount” (e.g., of an anti-cancer agent, such as an antagonist of PD-1, PD-L1 and/or CTLA4) may be an amount sufficient to increase the progression-free survival of the treated subject, for example by at least about 15%, at least about 20%, at least about 25%, at least about 50%, at least about 70%, at least about 75%, at least about 80%, at least about 90%, at least about 95%, at least 100%, at least 200%, at least 300%, at least 400%, or at least 500%, such as a progression-free survival increased by at least 3 months, at least 4 months, at least 6 months, at least 9 months, at least 1 year, at least 2 years, at least 3 years, at least 4 years, or at least 5 years (for example as compared to no administration of the therapeutic agent, or as compared to administration of the same agent, but in a subject having a tumor with normal or decreased expression of one or more ERVs provided herein). In some examples, combinations of these effects are achieved.

Endogenous Retrovirus (ERV): A family of viruses within the human genome with similarities to present day exogenous retroviruses. ERVs have been inherited by successive generations. Overall, ERVs constitute about 1% of the human genome. HERVs possess a similar genomic organization to present day exogenous retroviruses such as human immunodeficiency virus (HIV) and human T cell leukaemia virus (HTLV), and are composed of gag, pol, and env regions sandwiched between two long terminal repeats (LTRs). For example, see Nelson et al., Mol. Pathol. 56:11-18, 2003, herein incorporated by reference in its entirety. In some examples, high expression of ERV indicates that the ERV is immunogenic (also referred to herein as πERV), for example, as compared to a control, such as a tumor sample from a subject that is not responsive to at least one antagonist of an ICP-related molecule and/or a sample from a subject without a tumor. In some examples, high expression of an ERV indicates that the ERV expression is at least 1-fold, 2-fold, 3-fold, 4-fold, 5-fold, 8-fold, 10-fold, 15-fold, 50-fold, or 100-fold, or about 1-fold to 5-fold, 1-fold to 10-fold, 5-fold to 20-fold, 10-fold to 100-fold, or about 3-fold to 4-fold greater than the control.

Immune activation: Also referred to herein as immune checkpoint activation (ICA), upregulation of immune activation and/or immune checkpoint pathways (ICPs) are included (herein, immune checkpoint pathways include immune activation and/or immune checkpoint pathways). Immune activation refers to anti-tumor immune response. In some examples, one, two, or more indicators can be used, such as “ImmuneScore” (a marker of overall immune infiltration in tumor developed by Yoshihara, et al., Nat Commun, 4:2612, 2013, incorporated herein by reference in its entirety) and/or CD8A expression, which, in some examples, indicates CD8+ T cell infiltration, such as in a tumor, for example, a solid tumor. Immune checkpoint pathway upregulation includes upregulation of one or more ICPs, such as the PD-1 pathway (such as by upregulation of PD-1 and PD-L1 or PD-L2), the CTLA-4 pathway (such as by upregulation of CTLA-4 and CD80 or CD86), the BTLA-HVEM pathway, and/or LAG-3. In specific, non-limiting examples, ICA and/or ICP upregulation is indicated by upregulated PD-1 and PD-L1, CTLA-4 and CD80, BTLA and HVEM.

Increase or Decrease: A statistically significant positive or negative change, respectively, in quantity from a control value (such as a value representing the presence or absence of some condition, such as the absence of immunogenic ERVs, or πERVs). An increase is a positive change, such as an increase at least 50%, at least 100%, at least 200%, at least 300%, at least 400% or at least 500% as compared to the control value. A decrease is a negative change, such as a decrease of at least 20%, at least 25%, at least 50%, at least 75%, at least 80%, at least 90%, at least 95%, at least 98%, at least 99%, or at least 100% decrease as compared to a control value. In some examples the decrease is less than 100%, such as a decrease of no more than 90%, no more than 95%, or no more than 99%. In some examples, an increase is referred to herein as high expression. Increased or high expression can be measured using nucleic acid molecule amplification, such as using the ΔΔCt method (see, e.g., Schmittgen and Livak, Nature Protocols, 3(6): 1101-1108, 2008, incorporated by reference herein in its entirety).

Pharmaceutically acceptable carriers: The pharmaceutically acceptable carriers useful in this invention are conventional. Remington's Pharmaceutical Sciences, by E. W. Martin, Mack Publishing Co., Easton, Pa., 15th Edition (1975), describes compositions and formulations suitable for pharmaceutical delivery of a therapeutic agent, such as a PD1 antagonist, PD-L1 antagonist, and/or CTLA4 antagonist.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Subject: A vertebrate, such as a mammal, for example a human. Mammals include, but are not limited to, murines, simians, humans, farm animals, sport animals, and pets. In one embodiment, the subject is a non-human mammalian subject, such as a monkey or other non-human primate, mouse, rat, rabbit, pig, goat, sheep, dog, cat, horse, or cow. In some examples, the subject has a tumor, such as a cancer, that can be treated or analyzed using the methods disclosed herein. In some examples, the subject is a laboratory animal/organism, such as a mouse, rabbit, or rat.

Treating, Treatment, and Therapy: Any success or indicia of success in the attenuation or amelioration of an injury, pathology or condition, including any objective or subjective parameter such as abatement, remission, diminishing of symptoms or making the condition more tolerable to the patient, slowing in the rate of degeneration or decline, making the final point of degeneration less debilitating, improving a subject's physical or mental well-being, or prolonging the length of survival. The treatment may be assessed by objective or subjective parameters; including the results of a physical examination, blood and other clinical tests (such as imaging), and the like. In some examples, treatment with the disclosed methods results in a decrease in the number, volume, and/or weight of a tumor and/or metastases.

Tumor, neoplasia, malignancy or cancer: A neoplasm is an abnormal growth of tissue or cells which results from excessive cell division. Neoplastic growth can produce a tumor. The amount of a tumor in an individual is the “tumor burden” which can be measured as the number, volume, or weight of the tumor. A “non-cancerous tissue” is a tissue from the same organ wherein the malignant neoplasm formed, but does not have the characteristic pathology of the neoplasm. Generally, noncancerous tissue appears histologically normal. A “normal tissue” is tissue from an organ, wherein the organ is not affected by cancer or another disease or disorder of that organ. A “cancer-free” subject has not been diagnosed with a cancer of that organ and does not have detectable cancer.

Exemplary tumors, such as cancers, that can be treated or analyzed using the disclosed methods include solid tumors, such as breast carcinomas (e.g. lobular and duct carcinomas, such as a triple negative breast cancer), sarcomas, carcinomas of the lung (e.g., non-small cell carcinoma, large cell carcinoma, squamous carcinoma, and adenocarcinoma), mesothelioma of the lung, colorectal adenocarcinoma, stomach carcinoma, prostatic adenocarcinoma, ovarian carcinoma (such as serous cystadenocarcinoma and mucinous cystadenocarcinoma), ovarian germ cell tumors, testicular carcinomas and germ cell tumors, pancreatic adenocarcinoma, biliary adenocarcinoma, hepatocellular carcinoma, bladder carcinoma (including, for instance, transitional cell carcinoma, adenocarcinoma, and squamous carcinoma), renal cell adenocarcinoma, clear cell renal cell carcinoma, endometrial carcinomas (including, e.g., adenocarcinomas and mixed Mullerian tumors (carcinosarcomas)), carcinomas of the endocervix, ectocervix, and vagina (such as adenocarcinoma and squamous carcinoma of each of same), tumors of the skin (e.g., squamous cell carcinoma, basal cell carcinoma, malignant melanoma, skin appendage tumors, Kaposi sarcoma, cutaneous lymphoma, skin adnexal tumors and various types of sarcomas and Merkel cell carcinoma), esophageal carcinoma, carcinomas of the nasopharynx and oropharynx (including squamous carcinoma and adenocarcinomas of same), salivary gland carcinomas, brain and central nervous system tumors (including, for example, tumors of glial, neuronal, and meningeal origin), tumors of peripheral nerve, soft tissue sarcomas and sarcomas of bone and cartilage, head and neck squamous cell carcinoma, and lymphatic tumors (including B-cell and T-cell malignant lymphoma. In one example, the tumor is RCC. In one example, the tumor is ccRCC.

In specific, non-limiting examples, the methods can be used to treat clear cell renal cell carcinoma (ccRCC), breast cancer (such as ER+ HER2− breast cancer), colon cancer, or head and neck squamous cell cancer. In one specific, non-limiting example, the methods can be used to treat ccRCC,

### Methods of Detecting Immunogenic Endogenous Retroviruses (πERVs) and Methods of Treatment

### Methods of Detecting πERVs

Disclosed herein are methods of detecting immunogenic endogenous retroviruses (πERVs, such as endogenous retroviruses (ERVs) correlated with immune activation and immune checkpoint pathway upregulation, also referred to herein as ICA and ICP, respectively). In some examples, the methods include measuring expression of at least one endogenous retrovirus (ERV) in tumor sample from a subject responsive to at least one antagonist of an immune checkpoint pathway (ICP)-related molecule and measuring expression of at least one control sample from at least one of a tumor sample from a subject that is not responsive to at least one antagonist of an ICP-related molecule; and/or a sample from a subject without a tumor. In some examples, the methods include measuring expression of at least one endogenous retrovirus (ERV) in a tumor sample from a subject with a tumor and measuring expression of at least one control sample from at least one of a tumor sample from a subject that is not responsive to at least one antagonist of an ICP-related molecule and/or a sample from a subject without a tumor. In some examples, the ERV is considered an πERV if the ERV expression is at least 1-fold, at least 1.5 fold, at least 2-fold, at least 3-fold, at least 4-fold, at least 5-fold, at least 8-fold, at least 10-fold, at least 15-fold, at least 50-fold, or at least 100-fold, such as about 1-fold to 5-fold, 1-fold to 10-fold, 5-fold to 20-fold, 10-fold to 100-fold, or about 3-fold to 4-fold greater than a measured control sample or the amount of expression expected from a control sample. In some examples, the methods include measuring expression of at least one endogenous retrovirus (ERV) in a tumor sample from a subject, wherein at least one ERV is an πERV if the ERV expression is at least 1-fold, at least 2-fold, at least 3-fold, at least 4-fold, at least 5-fold, at least 8-fold, at least 10-fold, at least 15-fold, at least 50-fold, or at least 100-fold, or about 1-fold to 5-fold, 1-fold to 10-fold, 5-fold to 20-fold, 10-fold to 100-fold, or about 3-fold to 4-fold greater than a control sample, wherein the control sample comprises expression of the at least one ERV expected for at least one of a tumor sample from a subject that is not responsive to at least one antagonist of an ICP-related molecule and/or a sample from a subject without a tumor.

The methods include measuring expression of at least one endogenous retrovirus (ERV) in a tumor sample (such as tumor sample from clear cell renal cell carcinoma, breast cancer (for example, ER+ HER2− breast cancer), colon cancer, or head and neck squamous cell cancer, such as a tumor biopsy or fine needle aspirate) from a subject. In some examples, the sample is used directed, or filtered or concentrated or diluted before use. In some examples, nucleic acid molecules (such as DNA or RNA) are purified from the sample before use. In some examples, the tumor sample has increased or high expression of one or more ERVs, such as one or more of ERVK.1, ERVK.2, ERVK.3, ERVK.4, ERVK.5, ERVK.6, ERVK.7, ERVK.8, ERVK.9, ERVK.10, ERVK.11, ERVK.12, ERVK.13, ERVK.14, ERVK.15, ERVK.16, ERVK.17, ERVK.18, ERVK.19, ERVK.20, ERVK.21, ERVK.22, ERVK.23, ERVK.24, ERVK.25, ERV3.1, ERV3-2 (also referred to herein as ERV3.2), ERV3.3, ERV3.4, ERV3.5, ERV3.6, ERV3.7, ERV3.8, ERV9.1, ERVFRD.1, ERVFH21.1, ERVFC1.1, ERVFRD.2, ERVH48.1, ERVS71.1, ERVS71.2, ERVPABLB.1, and/or ERVMER61.1. (See, e.g., Mayer et al., “A revised nomenclature for transcribed human endogenous retroviral loci,” Mobile DNA, 2:7, 2011, incorporated herein by reference in its entirety). Measuring increased or high expression of ERVs can include measuring at least 1, at least 2, at least 3, at least 4, at least 5, at least 6, at least 7, at least 8, at least 9, at least 10, at least 20, at least 30, at least 40, at least 50, or at least 60 different ERVs, such as 1, 2, 3, 4, 5, 10, 15, 20, 25, 30, 35, 40, 45, or 50 different ERVs. Increased or high expression (such as nucleic acid expression) can include at 1-fold, 2-fold, 3-fold, 4-fold, 5-fold, 8-fold, 10-fold, 15-fold, 50-fold, or 100-fold, or about 1-fold to 5-fold, 1-fold to 10-fold, 5-fold to 20-fold, 10-fold to 100-fold, or about 3-fold to 4-fold greater expression (such as nucleic acid expression) than a control (for example, nucleic acid expression from a control sample), such as a control sample described herein.

Further, in some examples, measuring expression of at least one ERV includes measuring ERV nucleic acid expression through amplification of at least one ERV nucleic acid molecules. In specific, non-limiting examples, the ΔΔCt (also referred to as cycle threshold or Ct) method can be used (see, e.g., Schmittgen and Livak, Nature Protocols, 3(6): 1101-1108, 2008, incorporated by reference herein in its entirety). Expression of one or more housekeeping genes can also be measured to normalize the expression of the ERV measurement.

In specific, non-limiting examples, measuring ERV3-2 nucleic acid expression can include using:

(SEQ ID NO: 1)
5′-CAAGAGGCGGCATAGAAGCAA-3′
and
(SEQ ID NO: 2)
5′-GGAGAGTAGCTTGGGGTTTCA-3′;
(SEQ ID NO: 3)
5′-AGCCATTTACAAAGAAAGGGGAC-3′
and
(SEQ ID NO: 4)
5′-CTATGCCGCCTCTTGTCTGAT-3′;

or both.

In some examples, expression of one or more housekeeping genes (such as hypoxanthine-guanine phosphoribosyltransferase (HPRT1)) can also be measured to normalize the expression of the ERV measurement. Housekeeping genes include those whose expression is relatively similar (e.g., no more than a 10% difference, no more than a 5% difference, or no more than a 1% difference, such as a difference of about 0.01 to 10%) in the tumor sample as comparted to a corresponding control sample of the same tissue type (e.g., controls as disclosed herein; for example, expression is similar in normal kidney tissue and ccRCC, normal breast tissue and breast cancer tissue, normal colon tissue and colon cancer tissue, or normal head and neck tissue and head and neck squamous cell cancer tissue and/or expression is similar in ccRCC, breast cancer (such as ER+ HER2− breast cancer), colon cancer, or head and neck squamous cell cancer in a subject that is responsive and non-responsive to at least one antagonist of an immune checkpoint pathway (ICP)-related molecule). Other exemplary housekeeping genes whose expression can be measured and compared to one or more ERVs include beta actin (ACTB), glyceraldehyde-3-phosphate dehydrogenase (GAPDH), and phosphoglycerate kinase 1 (PGK1).

In specific, non-limiting examples, measuring HPRT1 expression can include using:

(SEQ ID NO: 5)
5′-GACACTGGCAAAACAATGCAGAC-3′
and
(SEQ ID NO: 6)
5′-TGGCTTATATCCAACACTTCGTGG-3′.

In specific, non-limiting examples, the methods include using amplification (such as by the ΔΔCt method) to measure expression of at least one ERV nucleic acid molecule in a tumor sample from a subject using primers encoded by SEQ ID NOS: 1-2 and/or 1-4 and to normalize the expression to HPRT nucleic acid molecules in the tumor sample from the subject using nucleic acids encoded by SEQ ID NOS: 5-6, wherein at least one ERV is an πERV if the ERV expression is 3-fold to 4-fold greater expression of the ERV in at least one control sample, such as at least one of a tumor sample from a subject that is not responsive to at least one antagonist of an ICP-related molecule and/or a sample from a subject without a tumor.

### Methods of Treatment

Further disclosed herein are methods of treating cancer. In examples, subjects with cancer are selected (such as subjects with clear cell renal cell carcinoma, breast cancer, colon cancer, or head and neck squamous cell cancer). Subjects having a tumor and identified as having an πERV (such as high or increased expression of at least one ERV) using the methods of detecting πERVs disclosed herein can receive an anti-cancer treatment, such as a therapeutically effective amount of an antagonist (such as an antibody) of PD-1, PD-L1, CTLA4, BTLA, HVEM, and/or LAG-3 (or combinations thereof).

Exemplary PD-1 antagonists and PD-L1 antagonists that can be used include an antibody (such as a mAb) that specifically binds and antagonizes PD-1 or PD-L1, such as atezolizumab, MPDL3280A, BNS-936558 (Nivolumab), pembrolizumab, Pidilizumab, CT011, AMP-224, AMP-514, MEDI-0680, BMS-936559, BMS935559, MEDI-4736, MPDL-3280A, MSB-0010718C, MGA-271, indoximod, epacadostat, BMS-986016, MEDI-4736, MEDI-4737, MK-4166, BMS-663513, PF-05082566 (PF-2566), lirilumab, and durvalumab. Exemplary CTLA4 antagonists that can be used include an antibody (such as a mAb) that specifically binds and antagonizes CTLA4, such as tremelimumab, ipilimumab, or both.

The subject treated with an antagonist of PD-1, PD-L1, CTLA4, BTLA, HVEM, and/or LAG-3 can receive one or more additional therapies, such as one or more of an effective amount of chemotherapy, an effective amount of radiotherapy (for example administration of radioactive material or energy (such as external beam therapy) to the tumor site to help eradicate the tumor or shrink it), an effective amount of a biologic (such as a therapeutic monoclonal antibody, ligand, or aptamer), and surgery (for example surgical resection of the cancer or a portion of it).

In one example, the subject is further treated with one or more chemotherapeutic agents. Chemotherapeutic agents include any chemical agent with therapeutic usefulness in the treatment of diseases characterized by abnormal cell growth, such as cancer. One of skill in the art can readily identify a chemotherapeutic agent of use (see for example, Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2^{nd} ed., © 2000 Churchill Livingstone, Inc; Baltzer, L., Berkery, R. (eds): Oncology Pocket Guide to Chemotherapy, 2nd ed. St. Louis, Mosby-Year Book, 1995; Fischer, D. S., Knobf, M. F., Durivage, H. J. (eds): The Cancer Chemotherapy Handbook, 4th ed. St. Louis, Mosby-Year Book, 1993; Chabner and Longo, Cancer Chemotherapy and Biotherapy: Principles and Practice (4th ed.). Philadelphia: Lippincott Willians & Wilkins, 2005; Skeel, Handbook of Cancer Chemotherapy (6th ed.). Lippincott Williams & Wilkins, 2003). Combination chemotherapy is the administration of more than one agent to treat cancer.

Examples of chemotherapeutic agents that can be used include alkylating agents, antimetabolites, natural products, or hormones and their antagonists. Examples of alkylating agents include nitrogen mustards (such as mechlorethamine, cyclophosphamide, melphalan, uracil mustard or chlorambucil), alkyl sulfonates (such as busulfan), nitrosoureas (such as carmustine, lomustine, semustine, streptozocin, or dacarbazine). Specific non-limiting examples of alkylating agents are temozolomide and dacarbazine. Examples of antimetabolites include folic acid analogs (such as methotrexate), pyrimidine analogs (such as 5-FU or cytarabine), and purine analogs, such as mercaptopurine or thioguanine. Examples of natural products include vinca alkaloids (such as vinblastine, vincristine, or vindesine), epipodophyllotoxins (such as etoposide or teniposide), antibiotics (such as dactinomycin, daunorubicin, doxorubicin, bleomycin, plicamycin, or mitocycin C), and enzymes (such as L-asparaginase). Examples of miscellaneous agents include platinum coordination complexes (such as cis-diamine-dichloroplatinum II also known as cisplatin), substituted ureas (such as hydroxyurea), methyl hydrazine derivatives (such as procarbazine), and adrenocrotical suppressants (such as mitotane and aminoglutethimide). Examples of hormones and antagonists include adrenocorticosteroids (such as prednisone), progestins (such as hydroxyprogesterone caproate, medroxyprogesterone acetate, and magestrol acetate), estrogens (such as diethylstilbestrol and ethinyl estradiol), antiestrogens (such as tamoxifen), and androgens (such as testosterone proprionate and fluoxymesterone).

Examples of chemotherapy drugs that can be used in the treatment include Adriamycin, Alkeran, Ara-C, BiCNU, Busulfan, CCNU, Carboplatinum, Cisplatinum, Cytoxan, Daunorubicin, DTIC, 5-fluoruracil (5-FU), Fludarabine, Hydrea, Idarubicin, Ifosfamide, Methotrexate, Mithramycin, Mitomycin, Mitoxantrone, Nitrogen Mustard, Taxol (or other taxanes, such as docetaxel), Velban, Vincristine, VP-16, while some more newer drugs include Gemcitabine (Gemzar), Herceptin, Irinotecan (Camptosar, CPT-11), Leustatin, Navelbine, Rituxan STI-571, Taxotere, Topotecan (Hycamtin), Xeloda (Capecitabine), Zevelin and calcitriol. Non-limiting examples of immunomodulators that can be used include AS-101 (Wyeth-Ayerst Labs.), bropirimine (Upjohn), gamma interferon (Genentech), GM-CSF (granulocyte macrophage colony stimulating factor; Genetics Institute), IL-2 (Cetus or Hoffman-LaRoche), human immune globulin (Cutter Biological), IMREG (from Imreg of New Orleans, La.), SK&F 106528, and TNF (tumor necrosis factor; Genentech).

Additional therapeutic agents that can be used in combination with an antagonist of PD-1, PD-L1, CTLA4, BTLA, HVEM, and/or LAG-3 include microtubule binding agents, DNA intercalators or cross-linkers, DNA synthesis inhibitors, DNA and/or RNA transcription inhibitors, antibodies, enzymes, enzyme inhibitors, gene regulators, angiogenesis inhibitors. These agents (which are administered at a therapeutically effective amount) and treatments can be used alone or in combination.

Microtubule binding agents refers to agents that interact with tubulin to stabilize or destabilize microtubule formation thereby inhibiting cell division. Examples of microtubule binding agents that can be used in conjunction with the disclosed therapies include, without limitation, paclitaxel, docetaxel, vinblastine, vindesine, vinorelbine (navelbine), the epothilones, colchicine, dolastatin 15, nocodazole, podophyllotoxin and rhizoxin. Analogs and derivatives of such compounds also can be used. For example, suitable epothilones and epothilone analogs are described in International Publication No. WO 2004/018478. Taxoids, such as paclitaxel and docetaxel, as well as the analogs of paclitaxel taught by U.S. Pat. Nos. 6,610,860; 5,530,020; and 5,912,264 can be used.

Suitable DNA and/or RNA transcription regulators, including, without limitation, actinomycin D, daunorubicin, doxorubicin and derivatives and analogs thereof also are suitable for use in combination with the disclosed therapies. DNA intercalators and cross-linking agents that can be administered to a subject include, without limitation, cisplatin, carboplatin, oxaliplatin, mitomycins, such as mitomycin C, bleomycin, chlorambucil, cyclophosphamide and derivatives and analogs thereof. DNA synthesis inhibitors suitable for use as therapeutic agents include, without limitation, methotrexate, 5-fluoro-5′-deoxyuridine, 5-fluorouracil (5-FU) and analogs thereof. Examples of suitable enzyme inhibitors include, without limitation, camptothecin, etoposide, formestane, trichostatin and derivatives and analogs thereof. Suitable compounds that affect gene regulation include agents that result in increased or decreased expression of one or more genes, such as raloxifene, 5-azacytidine, 5-aza-2′-deoxycytidine, tamoxifen, 4-hydroxytamoxifen, mifepristone and derivatives and analogs thereof.

The disclosed methods can further include administering to the subject a therapeutically effective amount of an immunotherapy. Non-limiting examples of immunomodulators that can be used include AS-101 (Wyeth-Ayerst Labs.), bropirimine (Upjohn), gamma interferon (Genentech), GM-CSF (granulocyte macrophage colony stimulating factor; Genetics Institute), IL-2 (Cetus or Hoffman-LaRoche), human immune globulin (Cutter Biological), IMREG (from Imreg of New Orleans, La.), SK&F 106528, and TNF (tumor necrosis factor; Genentech).

Non-limiting examples of anti-angiogenic agents include molecules, such as proteins, enzymes, polysaccharides, oligonucleotides, DNA, RNA, and recombinant vectors, and small molecules that function to reduce or even inhibit blood vessel growth. Examples of suitable angiogenesis inhibitors that can be used with the disclosed methods include, without limitation, angiostatin K1-3, staurosporine, genistein, fumagillin, medroxyprogesterone, suramin, interferon-alpha, metalloproteinase inhibitors, platelet factor 4, somatostatin, thromobospondin, endostatin, thalidomide, and derivatives and analogs thereof. For example, in some embodiments the anti-angiogenesis agent is an antibody that specifically binds to VEGF (e.g., Avastin, Roche) or a VEGF receptor (e.g., a VEGFR2 antibody). In one example the anti-angiogenic agent includes a VEGFR2 antibody, or DMXAA (also known as Vadimezan or ASA404; available commercially, e.g., from Sigma Corp., St. Louis, Mo.) or both. The anti-angiogenic agent can be bevacizumab, sunitinib, an anti-angiogenic tyrosine kinase inhibitors (TKI), such as sunitinib, xitinib and dasatinib. These can be used individually or in any combination.

Exemplary kinase inhibitors that can be used with the disclosed methods include Gleevac, Iressa, and Tarceva, sunitinib, sorafenib, anitinib, and dasatinib that prevent phosphorylation and activation of growth factors. Antibodies that can be used include Herceptin and Avastin that block growth factors and the angiogenic pathway. These can be used individually or in combination.

In some examples, the additional therapeutic agent administered is a biologic, such as a monoclonal antibody, for example, 3F8, Abagovomab, Adecatumumab, Afutuzumab, Alacizumab, Alemtuzumab, Altumomab pentetate, Anatumomab mafenatox, Apolizumab, Arcitumomab, Bavituximab, Bectumomab, Belimumab, Besilesomab, Bevacizumab, Bivatuzumab mertansine, Blinatumomab, Brentuximab vedotin, Cantuzumab mertansine, Capromab pendetide, Catumaxomab, CC49, Cetuximab, Citatuzumab bogatox, Cixutumumab, Clivatuzumab tetraxetan, Conatumumab, Dacetuzumab, Detumomab, Ecromeximab, Eculizumab, Edrecolomab, Epratuzumab, Ertumaxomab, Etaracizumab, Farletuzumab, Figitumumab, Galiximab, Gemtuzumab ozogamicin, Girentuximab, Glembatumumab vedotin, Ibritumomab tiuxetan, Igovomab, Imciromab, Intetumumab, Inotuzumab ozogamicin, Ipilimumab, Iratumumab, Labetuzumab, Lexatumumab, Lintuzumab, Lorvotuzumab mertansine, Lucatumumab, Lumiliximab, Mapatumumab, Matuzumab, Mepolizumab, Metelimumab, Milatuzumab, Mitumomab, Morolimumab, Nacolomab tafenatox, Naptumomab estafenatox, Necitumumab, Nimotuzumab, Nofetumomab merpentan, Ofatumumab, Olaratumab, Oportuzumab monatox, Oregovomab, Panitumumab, Pemtumomab, Pertuzumab, Pintumomab, Pritumumab, Ramucirumab, Rilotumumab, Rituximab, Robatumumab, Satumomab pendetide, Sibrotuzumab, Sonepcizumab, Tacatuzumab tetraxetan, Taplitumomab paptox, Tenatumomab, TGN1412, Ticilimumab (tremelimumab), Tigatuzumab, TNX-650, Trastuzumab, Tremelimumab, Tucotuzumab celmoleukin, Veltuzumab, Volociximab, Votumumab, Zalutumumab, or combinations thereof.

The therapy can be administered, in various examples, in a liquid dosage form, a solid dosage form, a suppository, an inhalable dosage form, an intranasal dosage form, in a liposomal formulation, a dosage form comprising nanoparticles, a dosage form comprising microparticles, a polymeric dosage form, or any combinations thereof. In certain cases, the therapy can be administered over a period of about 1 week to about 2 weeks, about 2 weeks to about 3 weeks, about 3 weeks to about 4 weeks, about 4 weeks to about 5 weeks, about 6 weeks to about 7 weeks, about 7 weeks to about 8 weeks, about 8 weeks to about 9 weeks, about 9 weeks to about 10 weeks, about 10 weeks to about 11 weeks, about 11 weeks to about 12 weeks, about 12 weeks to about 24 weeks, about 24 weeks to about 48 weeks, about 48 weeks or about 52 weeks, or longer. The frequency of administration of the therapy can be, in certain instances, once daily, twice daily, once every week, once every three weeks, once every four weeks (or once a month), once every 8 weeks (or once every 2 months), once every 12 weeks (or once every 3 months), or once every 24 weeks (once every 6 months).

### Clinical Response

Such methods can treat the tumor in the subject by reducing the volume or weight of the tumor, reducing the number of metastases, reducing the size or weight of a metastasis, or combinations thereof. In some examples a metastasis is cutaneous or subcutaneous. Thus, in some examples, administration of an antagonist of PD-1, PD-L1, CTLA4, BTLA, HVEM, and/or LAG-3 (alone or in combination with another anti-cancer therapy) treats a tumor having high or increased expression of an πERV in a subject by reducing the size or volume of the tumor having high or increased expression of an πERV by at least 10%, at least 20%, at least 30%, at least 40%, at least 50%, at least 60%, at least 70%, at least 75%, at least 80%, at least 90%, at least 95%, at least 98% or at least 99%, for example as compared to no administration of an antagonist of PD-1, PD-L1, CTLA4, BTLA, HVEM, and/or LAG-3 (or administration of an antagonist of PD-1, PD-L1, CTLA4, BTLA, HVEM, and/or LAG-3 to a subject having a tumor without high or increased expression of an πERV).

In some examples, administration of an antagonist of PD-1, PD-L1, CTLA4, BTLA, HVEM, and/or LAG-3 (alone or in combination with another anti-cancer therapy) treats a tumor having high or increased expression of an πERV in a subject by reducing the weight of the tumor by at least 10%, at least 20%, at least 30%, at least 40%, at least 50%, at least 60%, at least 70%, at least 75%, at least 80%, at least 90%, at least 95%, at least 98% or at least 99%, for example as compared to no administration of an antagonist of PD-1, PD-L1, CTLA4, BTLA, HVEM, and/or LAG-3 (or administration of an antagonist of PD-1, PD-L1, CTLA4, BTLA, HVEM, and/or LAG-3 to a subject having a tumor without high or increased πERV expression).

In some examples, administration of an antagonist of PD-1, PD-L1, CTLA4, BTLA, HVEM, and/or LAG-3 (alone or in combination with another anti-cancer therapy) treats a tumor having high or increased expression of an πERV in a subject by reducing the size or volume of a metastasis by at least 10%, at least 20%, at least 30%, at least 40%, at least 50%, at least 60%, at least 70%, at least 75%, at least 80%, at least 90%, at least 95%, at least 98% or at least 99%, for example as compared to no administration of an antagonist of PD-1, PD-L1, CTLA4, BTLA, HVEM, and/or LAG-3 (or administration of an antagonist of PD-1, PD-L1, CTLA4, BTLA, HVEM, and/or LAG-3 to a subject having a tumor without high or increased πERV expression).

In some examples, administration of an antagonist of PD-1, PD-L1, CTLA4, BTLA, HVEM, and/or LAG-3 (alone or in combination with another anti-cancer therapy) treats a tumor having high or increased expression of an πERV in a subject by reducing the number of metastases by at least 10%, at least 20%, at least 30%, at least 40%, at least 50%, at least 60%, at least 70%, at least 75%, at least 80%, at least 90%, at least 95%, at least 98% or at least 99% for example as compared to no administration of an antagonist of PD-1, PD-L1, CTLA4, BTLA, HVEM, and/or LAG-3 (or administration of an antagonist of PD-1, PD-L1, CTLA4, BTLA, HVEM, and/or LAG-3 to a subject having a tumor without high or increased πERV expression).

In some examples, administration of an antagonist of PD-1, PD-L1 and/or CTLA4 (alone or in combination with another anti-cancer therapy) treats a tumor having high or increased expression of an πERV in a subject by increasing the progression-free survival of the treated subject, for example by at least about 15%, at least about 20%, at least about 25%, at least about 50%, at least about 70%, at least about 75%, at least about 80%, at least about 90%, at least about 95%, at least 100%, at least 200%, at least 300%, at least 400%, or at least 500%, such as a progression-free survival increased by at least 3 months, at least 4 months, at least 5 months, at least 6 months, at least 7 months, at least 8 months, at least 9 months, at least 10 months, at least 11 months, at least 1 year, at least 2 years, at least 3 years, at least 4 years, or at least 5 years, for example as compared to no administration of an antagonist of PD-1, PD-L1, CTLA4, BTLA, HVEM, and/or LAG-3 (or administration of an antagonist of PD-1, PD-L1, CTLA4, BTLA, HVEM, and/or LAG-3 to a subject having a tumor without high or increased πERV expression). In some examples, combinations of these effects are achieved.

### EXAMPLES

Disclosed herein are RNA-seq and ERV expression (Rooney et al., Cell, 160(1-2):48-61, 2015) data for tumors (N=472 for ccRCC, N=4,438 for 20 other cancers) from The Cancer Genome Atlas (TCGA) demonstrating that elevated expression of a set of πERVs is associated with ICA (as determined using gene expression) in several solid cancers and is particularly strikingly in ccRCC. Also disclosed herein, expression of an πERV, ERV3-2, which is associated with responsiveness to single-agent PD-1/PD-L1 blockade in a cohort of metastatic ccRCC patients (N=24). This disclosure indicates that broad transcriptional activation of πERVs, present in a subset of tumors, induces anti-tumor immune response and subsequent up-regulation of immune checkpoint pathways, making such tumors sensitive to ICB. Expression of seven members of the ERVK family is correlated with the expression of cytotoxins GZMA and PRF1 in ccRCC (Rooney et al., Cell, 160(1-2):48-61, 2015), and an association between ERV3-2 expression and response to ICB in a small cohort of 24 metastatic ccRCC patients is disclosed herein. Although πERV-high tumors in ccRCC have poor prognosis under standard therapy, such tumors may have significantly improved outcomes with ICB.

In addition to CD8+ T-cells, M1-macrophages were more abundant, and M2-macrophages were less abundant in πERV-high ccRCC tumors compared to πERV-low tumors in TCGA. Because M1-macrophages have tumor inhibitory properties and M2-macrophages are indicated as tumor promoting (Yuan et al., Sci Rep., 5:14273, 2015), πERV-high tumors are indicated as having a relatively favorable immune microenvironment. Consistently, such enrichment of M1-polarization in macrophages has been observed in both mutation burden-associated (Mehnert et al., J Clin Invest., 126(6):2334-40, 2016) (Panda et al., JCO Precis Oncol., 2017) and exogenous virus-associated (Panda et al., J Natl Cancer Inst., 110(3):316-320, 2018) immunogenicity in other cancers.

Disclosed herein, overall ERV expression in tumors correlates with the expression of genes involved in histone methylation and chromatin regulation in multiple cancers, including ccRCC. These data indicate that ERV expression is induced by a dysfunction in chromatin organization, which plays a key role in the normal epigenetic silencing of ERVs. An enrichment of BAP1 mutations was observed in the πERV-high subtype of KIRC, indicating that BAP1 dysfunction leads to chromatin abnormalities, resulting in πERV expression. BAP1 is a de-ubiquitinase and is known to functionally associate with chromatin regulating complexes (LaFave et al., Nat Med., 21(11):1344-9, 2015). Further, loss of PBRM1 has been associated with response to non-first-line ICB in pre-treated ccRCC (Miao et al., Science, 359(6377):801-806, 2018). No enrichment of PBRM1 mutations was observed in the πERV-high subtype of KIRC, the data disclosed herein indicate a link between changes in chromatin and an immune phenotype. These chromatin modifiers exist with an obligate loss of heterozygosity in ccRCC due to the characteristic chromosome 3p deletion, and extrapolation to other tumor types, such as papillary RCC or bladder cancers harboring these mutations, is complex. ccRCC tumors are also relatively enriched in frameshift mutations caused by small insertion/deletions (Turajlic et al., Lancet Oncol., 18(8):1009-1021, 2017).

In addition to ccRCC, overexpression of πERVs was also associated with ICA in ER+/HER2− breast cancer, colon cancer, and head-neck squamous-cell cancer. πERV overexpression overlapped somewhat with genomic features potentially associated with ICA in these cancers, such as APOBEC mutagenesis (Roberts et al., Nat Genet., 45(9):970-6, 2013) in ER+/HER2− breast cancer, microsatellite instability in colon cancer, and HPV infection in head-neck squamous cancer, but πERV overexpression was associated with ICA independently of these features. Moreover, expression of ERV3-2 in particular was correlated with ICA in 11 solid cancers, including the four noted above. Thus, like hypermutation or exogenous viral expression in tumors, πERV expression in tumors is a mechanism of ICA operation in multiple solid cancers. In summary, the data disclosed herein indicate that a set of πERVs are broadly overexpressed in a subset of tumors and are associated with ICA in ccRCC.

### Example 1—Methods

Processing of TCGA RNA-seq Data:

RNAseqV2 scaled estimates were obtained from Broad Genome Data Analysis Centers (GDAC) and TCGA data portal. The data were median-adjusted so that the median scaled estimate was unity in each sample and were then used as input for the ESTIMATE (Yoshihara et al., Nat Commun., 4:2612, 2013) and CIBERSORT (Newman et al., Nat Methods, 12(5):453-7, 2015) algorithms to quantify the level of immune infiltration in tumor (“ImmuneScore”) as well as the composition of infiltrating leukocytes. Only unambiguous (P<0.05) CIBERSORT outputs were retained. For the remainder of the analysis, the median-adjusted data (x) was log-transformed to y=log 2(1+1023 x), so that the lowest possible expression was 0 units, and the median expression was 10 units in each sample.

Source of the Remaining TCGA Data:

ERV expression data for a large subset of the TCGA cohort was downloaded from a recent study (Rooney et al., Cell, 160(1-2):48-61, 2015) that quantified normalized expression levels of 66 transcribed ERVs (Mayer et al., Mob DNA, 2(1):7, 2011) by direct remapping from the raw RNA-seq data. Only tumors for which both mRNA and ERV expression data were available were included in the analyses (N=472 for ccRCC, N=4,438 for 20 other cancers). ERBB2 focal copy number data and ESR1 mRNA expression data obtained from Broad GDAC were used to classify breast cancer into clinical subtypes (ER+/HER2−, ER−/HER2−, HER2+), and each subtype was analyzed separately. Clinical and mutation data were downloaded from cBioPortal, APOBEC enrichment data was compiled from P-MACD (Roberts et al., Nat Genet., 45(9):970-6, 2013) files from Broad GDAC, microsatellite status and HPV status were compiled from auxiliary files from TCGA Data Portal.

Statistical Methods:

P-values reported in bar plots, boxplots, and Kaplan-Meier plots are from Fisher's exact test, Wilcoxon rank-sum test, and log-rank tests, respectively. Statistical significance was assessed at P<0.05 in two-sided tests. False discovery rates used the Benjamini-Hochberg procedure, and hazard ratios are from univariate cox regression. Correlated/anticorrelated was defined as Spearman Rho [symbol] 0 and P<0.05, whereas uncorrelated was defined as P≥0.05.

Search for Potentially Immunogenic ERVs:

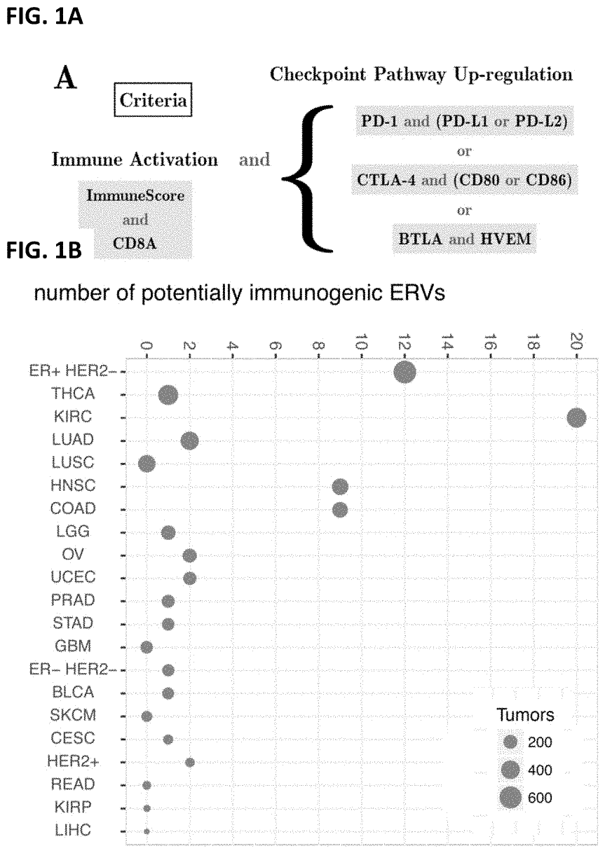

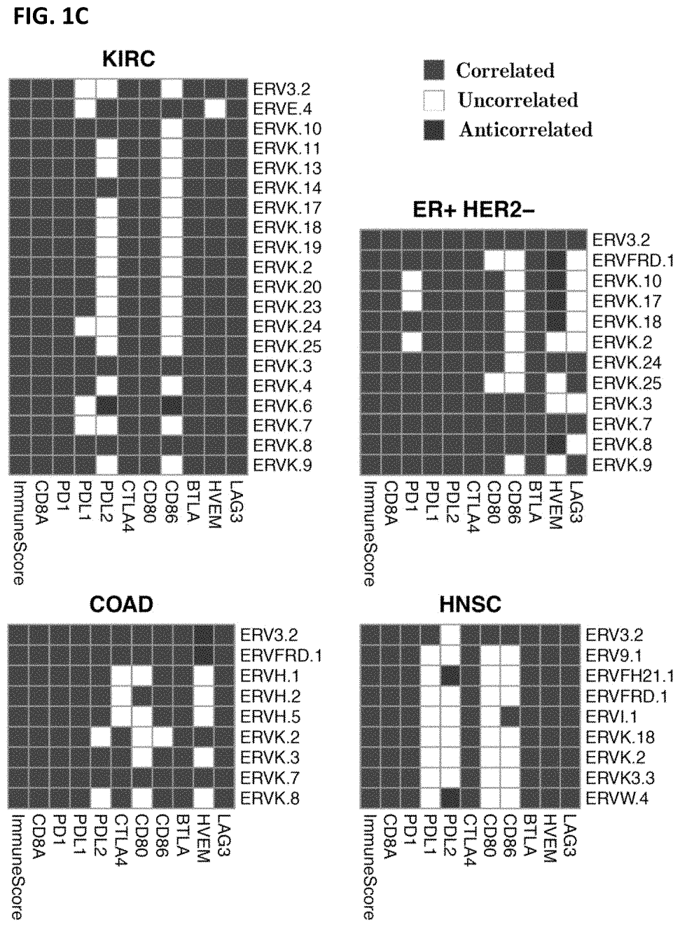

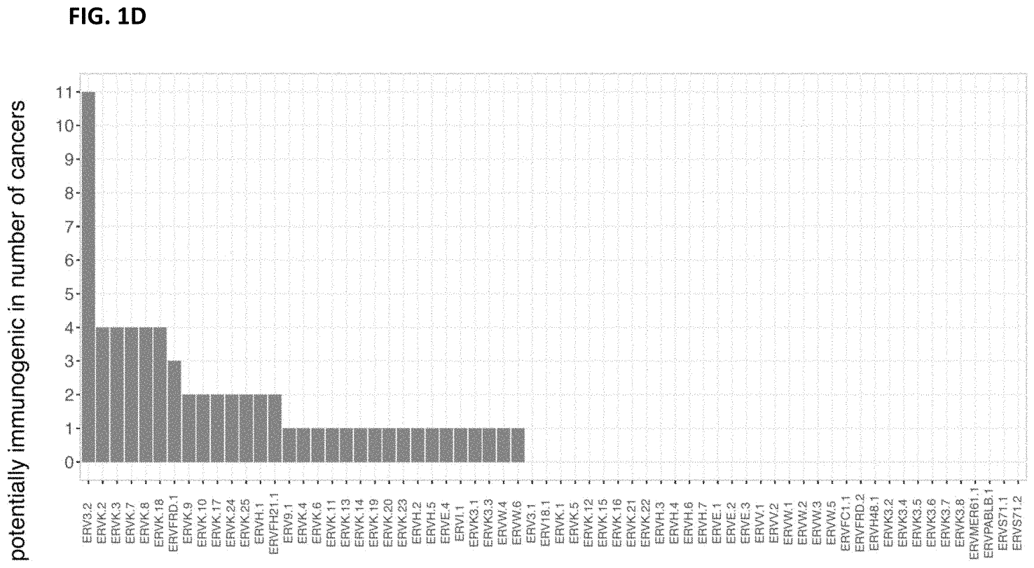

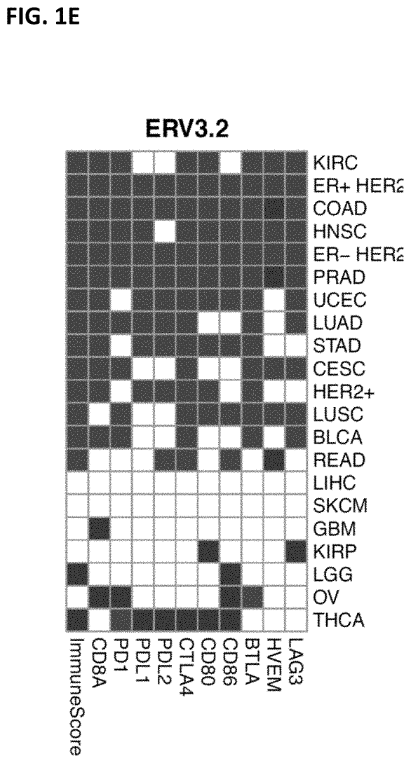

An ERV was considered potentially immunogenic if its expression was correlated with both immune activation and checkpoint pathway up-regulation (FIG. 1A). The immune activation criterion was considered satisfied if the expression of an ERV correlated with immune infiltration (“ImmuneScore” from ESTIMATE (Yoshihara et al., Nat Commun, 4:2612, 2013)) and mRNA expression of CD8A (marker of CD8+ T-cell infiltration). The checkpoint pathway up-regulation criterion was considered satisfied if the expression of an ERV correlated with either the PD-1 pathway (i.e., PD-1 and at least one of its ligands), the CTLA-4 pathway (i.e., CTLA-4 and at least one of its ligands), or the BTLA-HVEM pathway. In each cancer type, whether any of the 66 transcribed ERVs (Mayer et al., Mob DNA, 2(1):7, 2011) satisfied both immune activation and checkpoint pathway up-regulation criteria was examined.

Gene Expression-Based Enrichment Analyses:

Defining overall ERV expression as the total fraction of RNA-seq reads that mapped to the 66 transcribed ERVs (Mayer et al., Mob DNA, 2(1):7, 2011), genes whose expressions correlated (Spearman Rho >0, P<0.05) with overall ERV expression in each cancer type were identified. Six hundred fifty-seven genes satisfied this criterion in ccRCC (KIRC), ER+/HER2−, and COAD. An enrichment analysis of these genes was performed using the ToppGene (Chen et al., Nucleic Acids Res., 37(Web Server issue):W305-11, 2009) suite to identify enriched Gene Ontology terms.

Patient Samples in the Validation Cohort:

Formalin-fixed paraffin embedded (FFPE) metastatic ccRCC samples from patients treated with single-agent PD-1/PD-L1 blockade were collected at the Vanderbilt-Ingram Cancer Center. All tissues were evaluated by a pathologist using hematoxylin and eosin staining, and only the samples containing ≥70% tumor cells were included in this study. The cohort included 13 patients who experienced partial response (PR) with progression-free survival of at least six months and 11 patients who demonstrated immediate progressive disease (PD).

Quantification of ERV3-2 Expression in the Validation Cohort:

Total RNA isolation was performed using the RNAeasy FFPE Kit (Qiagen). DNAse treatment was performed during RNA isolation using RNase-free DNase I (Qiagen). RNA quality and concentration were assessed using a NanoDrop ND-1000 spectrophotometer (NanoDrop Technologies, Wilmington, Del., USA). First-strand cDNA synthesis was performed using 250 ng total RNA, random hexamers, and the SuperScript IV Reverse Transcriptase Kit (Life Technologies). Real-time quantitative PCRs (RT-qPCRs) were performed on a CFX96 Touch Real-Time PCR Detection System (Bio-Rad) using the SYBRgreen master mix reagent (Life Technologies). Primer sequences for ERV3-2 and HPRT1 (fwd: 5′-GACACTGGCAAAACAATGCAGAC-3′ (SEQ ID NO: 5), rev: 5′-TGGCTTATATCCAACACTTCGTGG-3′ (SEQ ID NO: 6)) were designed using PrimerBank (Wang et al., Nucleic Acids Res., 40(Database issue):D1144-9, 2012). ERV3-2 was quantified using two different primers: ERV3-2_1 (fwd: 5′-CAAGAGGCGGCATAGAAGCAA-3′ (SEQ ID NO: 1), rev: 5′-GGAGAGTAGCTTGGGGTTTCA-3′ (SEQ ID NO: 2)) and ERV3-2_2 (fwd: 5′-AGCCATTTACAAAGAAAGGGGAC-3′ (SEQ ID NO: 3), rev: 5′-CTATGCCGCCTCTTGTCTGAT-3′ (SEQ ID NO: 4)). All analyses were performed in triplicate, and relative RNA levels were determined using HPRT1 as an endogenous internal control. A HeLa control RNA sample was included for inter-plate calibration. ERV3-2 expression level was calculated using the ΔΔCt method.

### Example 2—πERVs are Abundant in Four Solid Cancers from TCGA

To identify πERVs (details in Example 1), 21 solid cancer types from TCGA were evaluated for correlation between expression levels (Rooney et al., Cell, 160(1-2):48-61, 2015) of 66 transcribed ERVs (Mayer et al., Mob DNA, 2(1):7, 2011) and RNA-seq based evidence of local immune checkpoint activation. As shown in FIG. 1A, ICA criteria included markers of immune activation, namely overall immune infiltration (“ImmuneScore” from ESTIMATE (Yoshihara et al., Nat Commun., 4:2612, 2013)) and expression of the cytotoxic T-cell marker CD8A, and markers of checkpoint pathway up-regulation, namely expression of genes in the PD-1, CTLA-4, and BTLA-HVEM pathways.

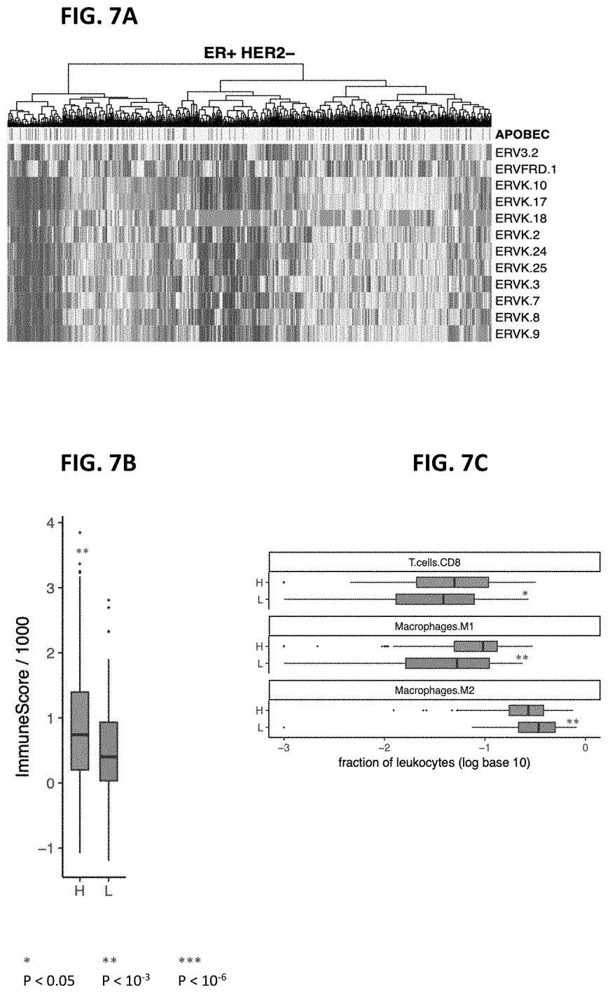

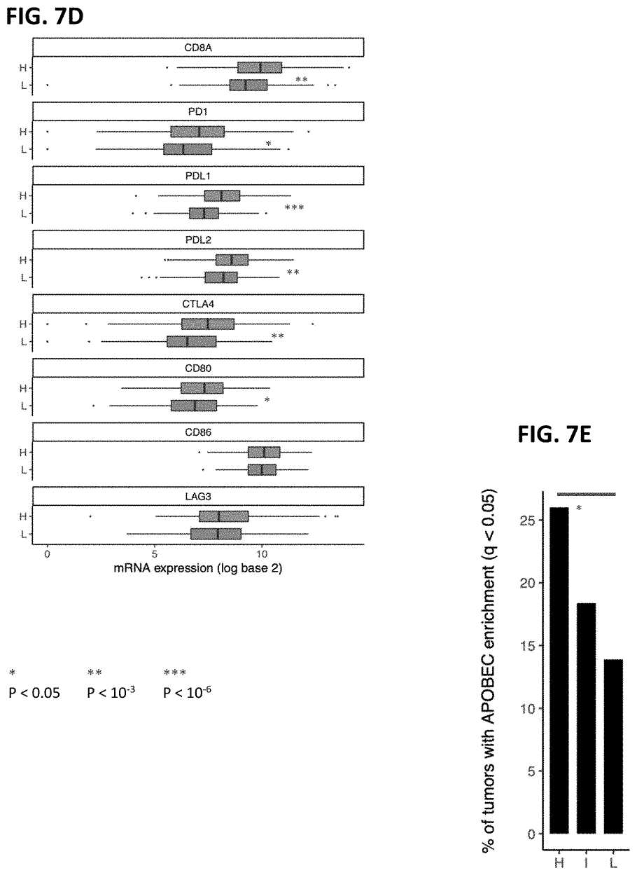

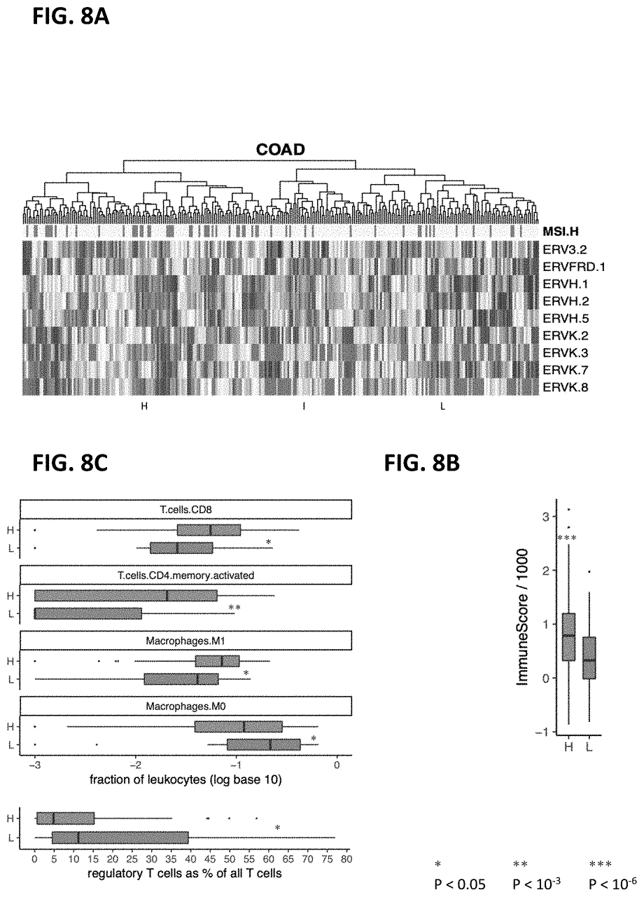

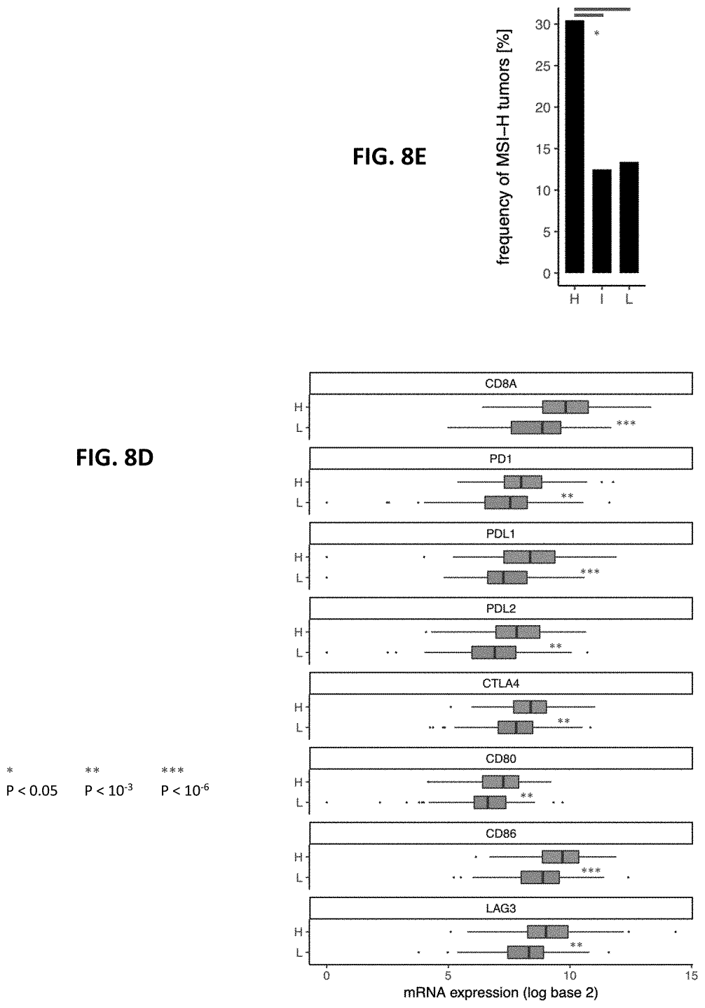

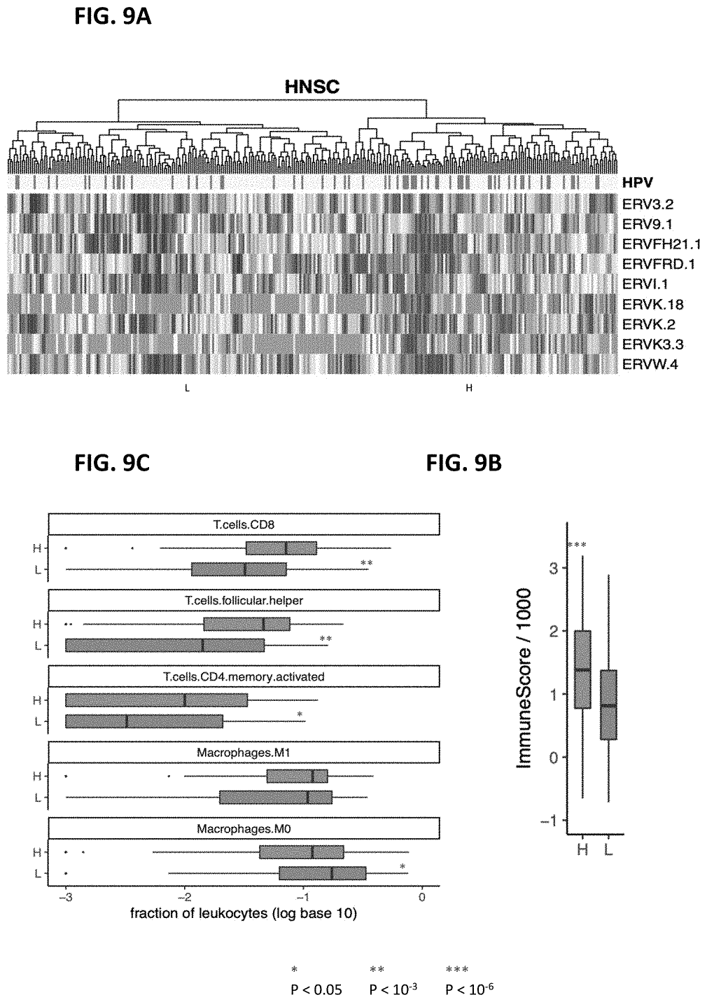

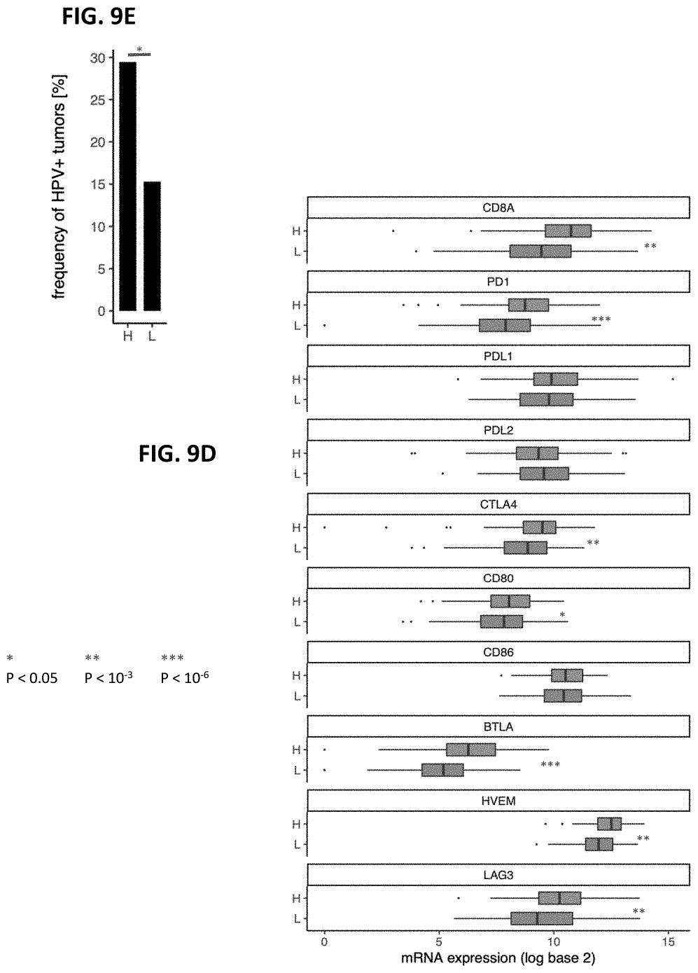

Results of this analysis (summarized in FIG. 1B, and detailed in FIGS. 7A-9E) showed that πERVs are abundant in four cancers, namely ccRCC (KIRC), ER+/HER2− breast cancer (ER+ HER2−), colon cancer (COAD), and head-neck squamous-cell cancer (HNSC). In these cancers, expression of nine to twenty πERVs correlated with ICA compared to background levels from zero to two πERVs in other cancers (FIG. 1B). πERVs were most abundant in ccRCC (KIRC), where expression of 20 πERVs correlated with ICA, most of which (18/20) were members of the ERVK family (FIG. 1C). While most ERVs were identified as πERVs in zero or one cancer type and several were identified across multiple diseases, ERV3-2 was identified as a πERV in 11 different solid cancers (FIGS. 1D-1E), including the four cancers named above.

### Example 3—Expression of πERVs Define Subtypes with Differential ICA in ccRCC

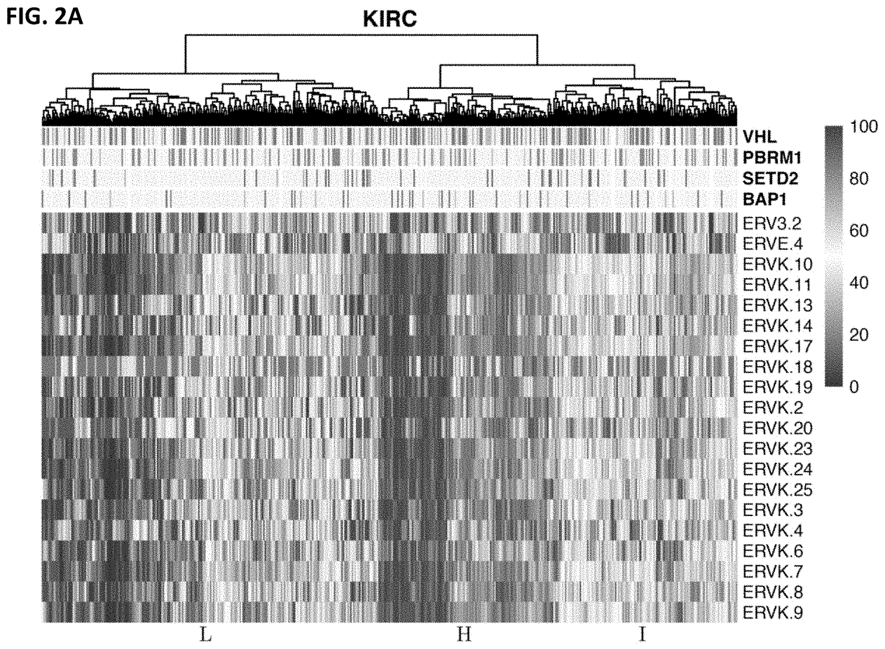

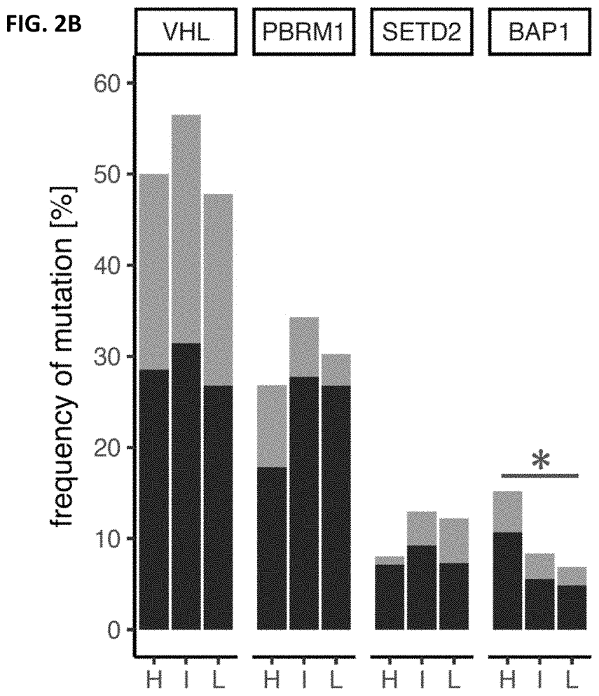

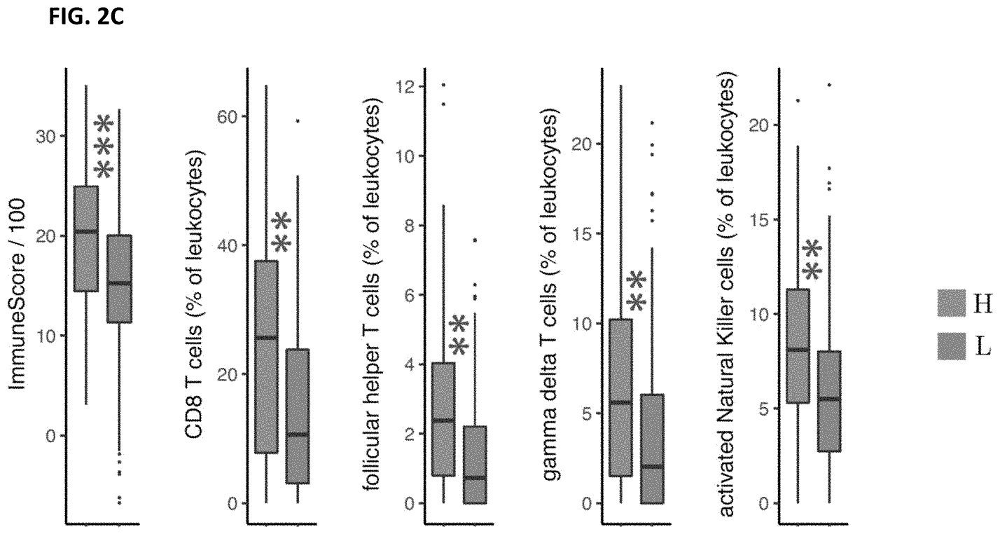

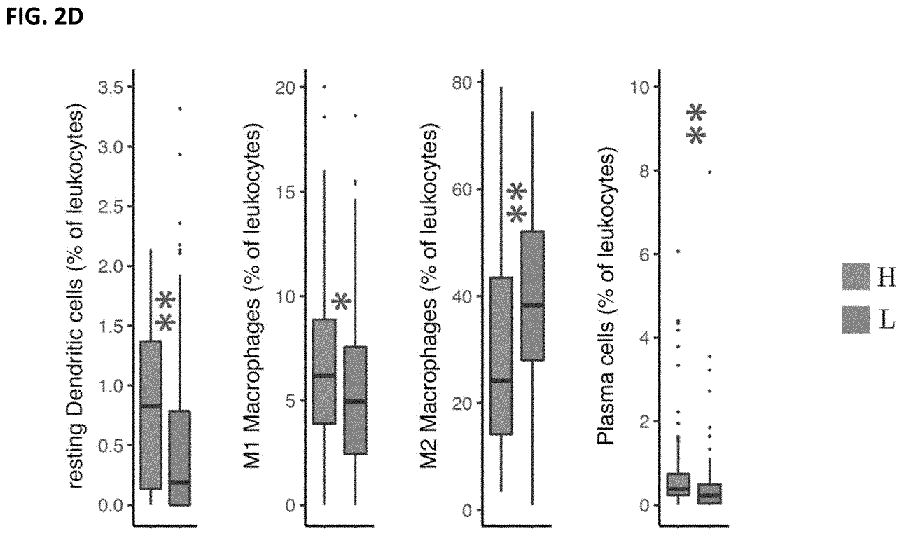

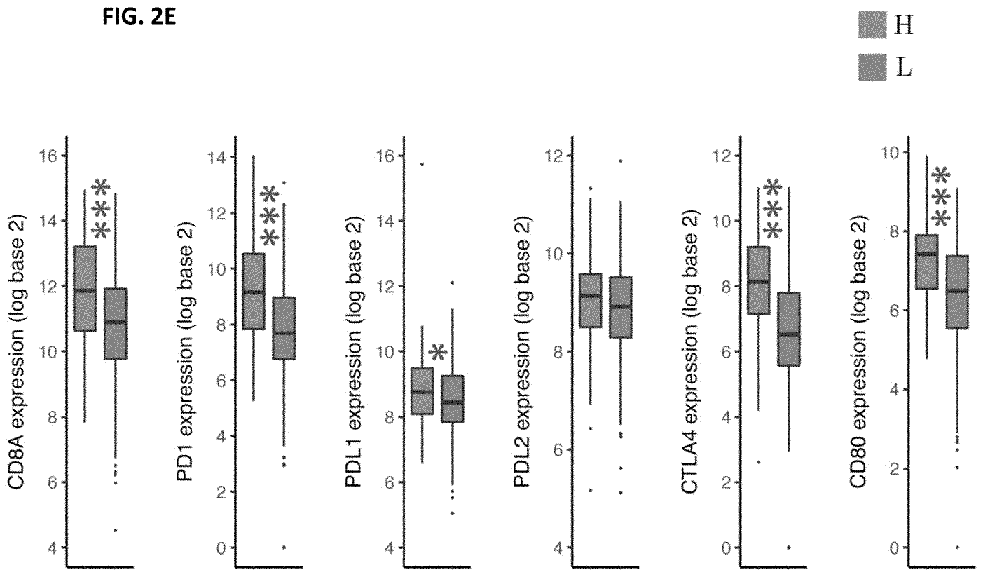

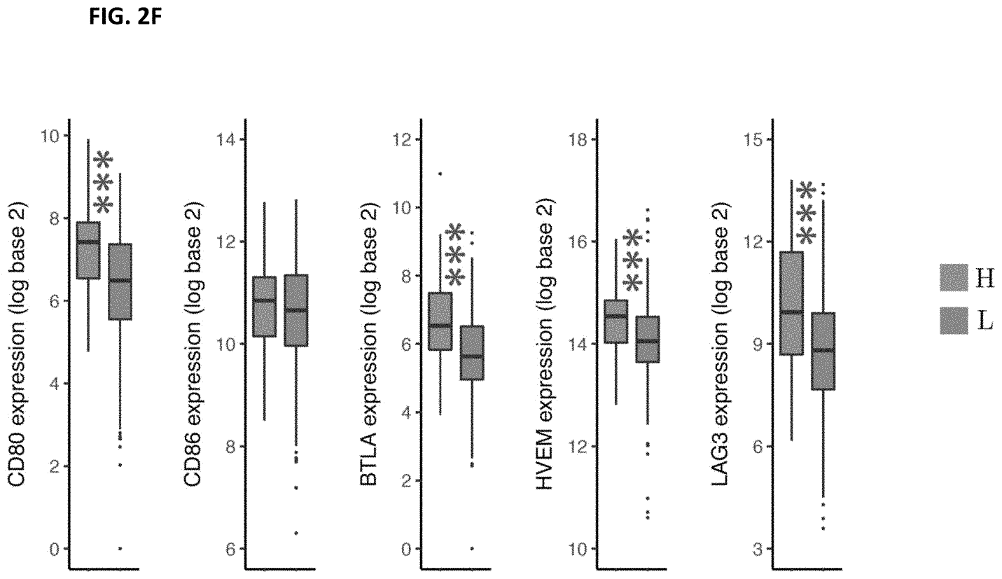

In the TCGA ccRCC (KIRC) cohort, the 20 πERVs were mostly co-expressed. Hierarchical clustering of tumors by percentile expression of these 20 πERVs identified three distinct subtypes corresponding to high (H), intermediate (I) and low (L) expression of πERVs (FIG. 2A). Loss-of-function mutations in chromatin regulatory genes (including PBRM1, SETD2, and BAP1) are frequently observed in ccRCC (Cancer Genome Atlas Research Network. Nature, 499(7456):43-9, 2013) and an association between PBRM1 loss and response to non-first-line ICB in pre-treated ccRCC has been observed (Miao et al., Science, 359(6377):801-806, 2018). Thus, the frequency of mutation of these genes in the three πERV expression-based subtypes were examined in KIRC. As shown in FIG. 2B, although there was no significant enrichment of VHL, PBRM1, or SETD2 mutations in the πERV-high ccRCC tumors, there was a statistically significant enrichment of BAP1 mutations in the πERV-high tumors compared to πERV-low tumors (15.2% vs 6.8%, Odds Ratio 2.44 [95% CI: 1.15-5.16], P=0.028).

πERV-high tumors also had significantly higher immune infiltration (FIGS. 2C-2D), a significantly higher fraction of CD8+ T-cells among tumor infiltrating leukocytes (FIGS. 2C-2D) and significantly higher mRNA expression of the cytotoxic T-cell marker CD8A (FIGS. 2E-2F) compared to πERV-low tumors, indicating immune activation in the πERV-high tumors. In addition, follicular-helper T-cells, γδ T-cells, activated NK cells, resting dendritic cells, and plasma cells constituted a significantly higher fraction of tumor infiltrating leukocytes in the πERV-high tumors compared to πERV-low tumors (FIGS. 2C-2D). Furthermore, M1-macrophages were more abundant in πERV-high tumors, whereas M2-macrophages were more abundant in πERV-low tumors (FIGS. 2C-2D).

πERV-high tumors also had significantly higher mRNA expression of several checkpoint genes (PD-1 and PD-L1, CTLA-4 and CD80, BTLA and HVEM, LAG-3) compared to πERV-low tumors (FIGS. 2E-2F), indicating checkpoint pathway up-regulation in πERV-high tumors. Expression levels of most πERVs correlated with expression levels of PD-L1 but not PD-L2, and CD80 but not CD86 (FIG. 1C). Consistently, in contrast to PD-L1 and CD80, PD-L2 and CD86 were not differentially expressed in πERV-high versus πERV-low tumors (FIGS. 2E-2F).

### Example 4—Expression of ERV3-2 Predicts Response to ICB in ccRCC

ccRCC (KIRC) showed the strongest evidence of πERV associated ICA among the 21 solid cancers (FIG. 1B), and ERV3-2 showed the most consistent correlation with ICA among the 66 transcribed ERVs (FIGS. 1D-1E). Thus, the expression of ERV3-2 in ccRCC tumors was examined as a predictor of response to ICB.

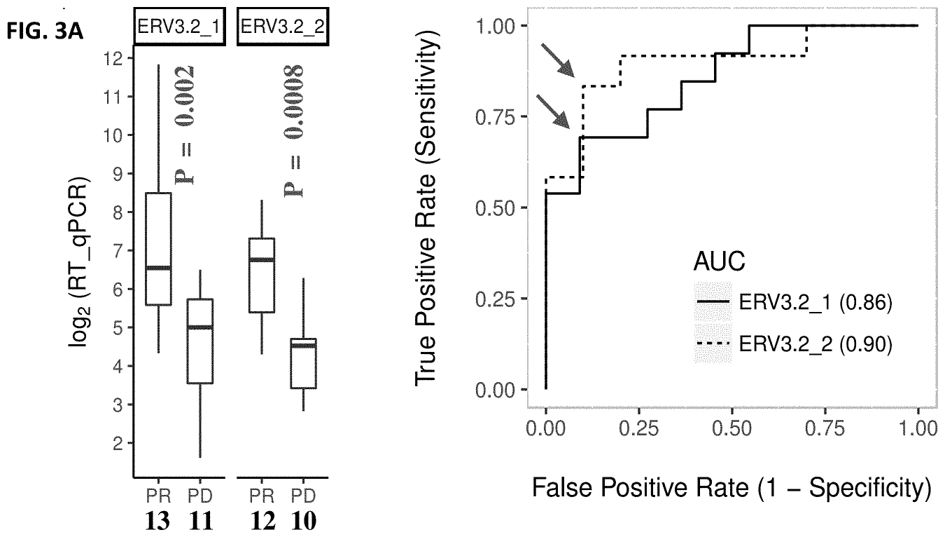

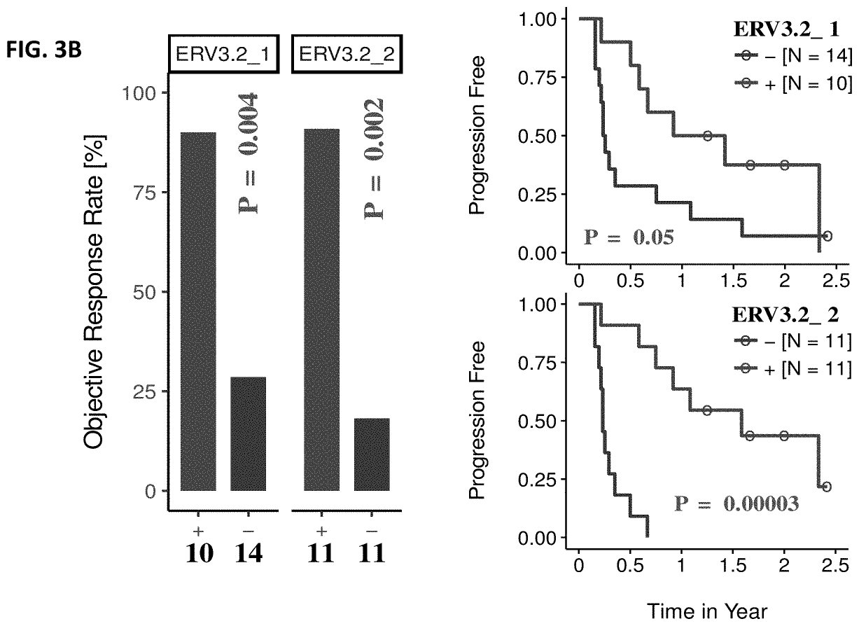

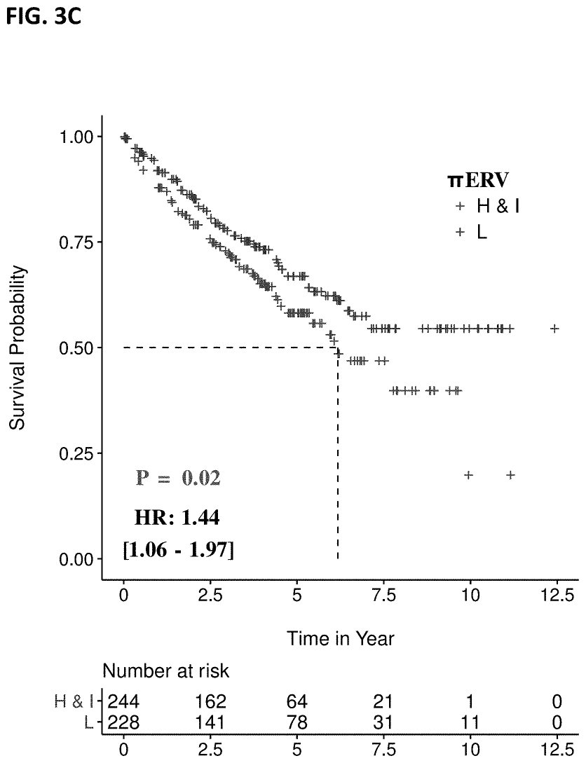

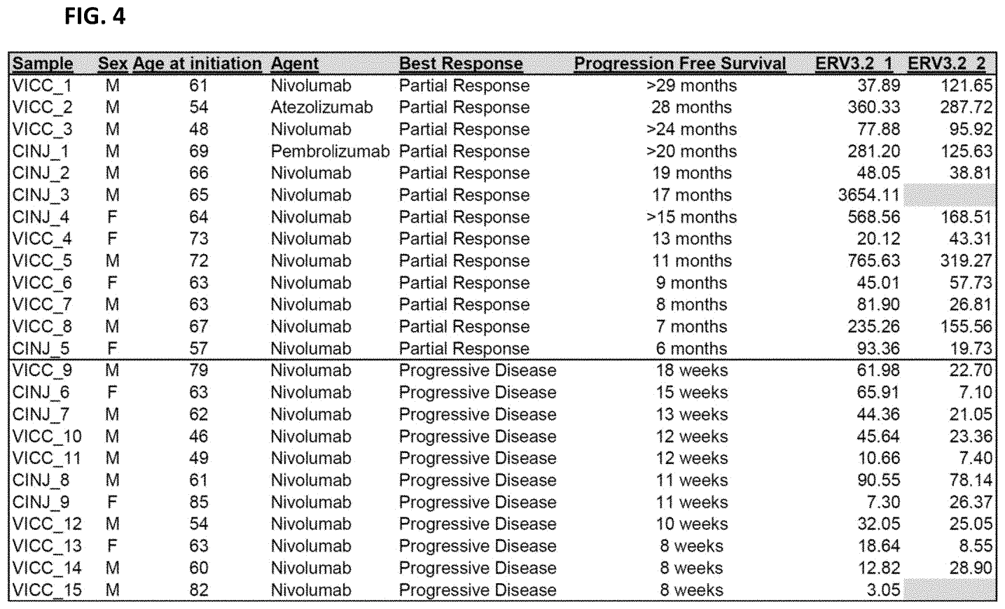

The ERV3-2 RNA level was measured by RT-qPCR in tumors of 24 metastatic ccRCC patients treated with single-agent PD-1/PD-L1 antibody (FIG. 4). The cohort consisted of 13 patients with partial response (PR) with progression free survival six months or longer and 11 non-responders who had immediate progressive disease (PD). The ERV3-2 RNA level was quantified using two different primers (details in Example 1), referred to here as ERV3-2_1 and ERV3-2_2 (1 responder and 1 non-responder failed in case of ERV3-2_2). ERV3-2 RNA levels, as measured by either primer, were significantly higher (P=0.002 for ERV3-2_1, P=0.0008 for ERV3-2_2) in tumors from responders compared to tumors from non-responders and was an excellent predictor of response to ICB (area under ROC curve: 0.86 for ERV3-2_1, 0.90 for ERV3-2_2) in the preliminary analysis based on this collection of samples (FIG. 3A).

To confirm consistent results, patients were classified into ERV3-2+ and ERV3-2− groups based on whether their tumor had higher or lower expression of ERV3-2 compared to the optimal cut-off inferred from the ROC curves (marked by a green arrow in FIG. 3A). As shown in FIG. 3B, ERV3-2+ patients had a significantly higher objective response rate compared with ERV3-2− patients for both ERV3-2_1 (90.0% vs 28.6%, Odds Ratio 22.5 [95% CI: 2.1-240.5], P=0.004) and ERV3-2_2 (90.9% vs 18.2%, Odds Ratio 45.0 [95% CI: 3.5-584.3], P=0.002). Consistently, ERV3-2+ patients had a longer progression-free survival (FIG. 3B) compared to ERV3-2− patients for both ERV3-2_1 (Hazard Ratio 0.53 [95% CI: 0.27-1.02], P=0.05) and ERV3-2_2 (Hazard Ratio 0.15 [95% CI: 0.05-0.44], P=0.00003).

These results indicate that ccRCC tumors with sufficiently high expression of ERV3-2 is enriched in the pool of tumors sensitive to ICB. Notably, patients with πERV-high and πERV-intermediate tumors have significantly shorter overall survival (Hazard Ratio 1.44 [95% CI: 1.06-1.97], P=0.02) compared to patients with πERV-low tumors under standard therapy in ccRCC (FIG. 3C). This is consistent with the enrichment of BAP1 mutations in the πERV-high subtype of ccRCC (FIG. 2B), as BAP1 mutations are associated with poor prognosis in ccRCC (Kapur et al., Lancet Oncol. 2013 February; 14(2):159-167).

### Example 5—Expression of πERVs Define Subtypes with Differential ICA in ER+/HER2− Breast Cancer, Colon Cancer, and Head-Neck Squamous-Cell Cancer

Like KIRC, πERV-expression-based subtypes were also observed in ER+/HER2−, COAD, and HNSC (FIGS. 7A, 8A, and 9A) in TCGA data. Thus, a clearly identifiable subset of tumors displays broad transcriptional activation of πERVs in these four cancers.

Similar to KIRC, the πERV-high tumors had significantly higher immune infiltration, a significantly higher fraction of CD8+ T-cells in infiltrating leukocytes, and significantly higher mRNA expression of the cytotoxic T-cell marker CD8A compared to πERV-low tumors in these three cancers (FIGS. 7B-7D, 8B-8D, and 9B-9D), indicating immune activation in the πERV-high subtype in all four cancers. Furthermore, M1-macrophages were more abundant in the πERV-high subtype of ER+ HER2− and COAD (but not HNSC), whereas M2 or M0-macrophages were more abundant in the πERV-low subtype of all three cancers (FIGS. 7C, 8C, and 9C). Additionally, activated memory CD4+ T-cells in COAD and HNSC as well as follicular-helper T-cells in HNSC comprised a significantly higher fraction of infiltrating leukocytes, and regulatory T-cells in COAD comprised a significantly lower fraction of T-cells in the πERV-high tumors compared with the πERV-low tumors (FIGS. 7C, 8C, and 9C).

As in KIRC, πERV-high tumors had significantly higher mRNA expression of checkpoint genes in the PD-1 and CTLA-4 pathways compared to πERV-low tumors in ER+ HER2− and COAD (FIGS. 7D, 8D, and 9D), indicating checkpoint pathway up-regulation in the πERV-high tumors in these cancers. Unlike ER+ HER2− and COAD, expression of most πERVs in HNSC did not correlate with the expressions of the ligands of PD-1 and CTLA-4 (FIG. 1C). Consistently, PD-L1 and PD-L2 were not differentially expressed in πERV-high versus πERV-low tumors in HNSC. Instead, the BTLA-HVEM pathway and LAG-3 were up-regulated in πERV-high tumors in HNSC (FIGS. 7D, 8D, and 9D).

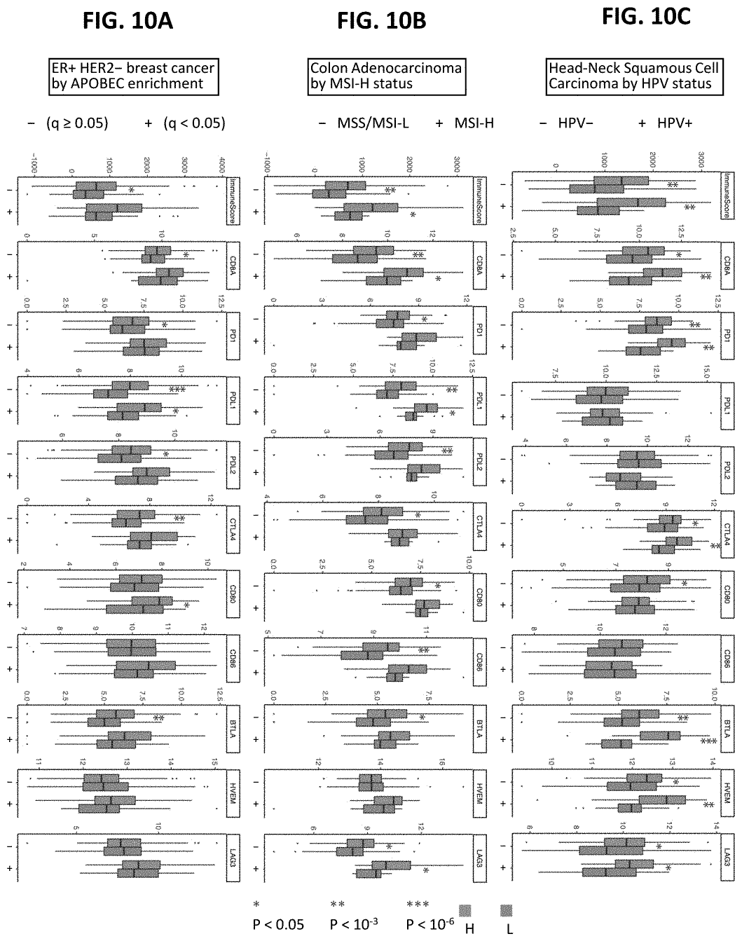

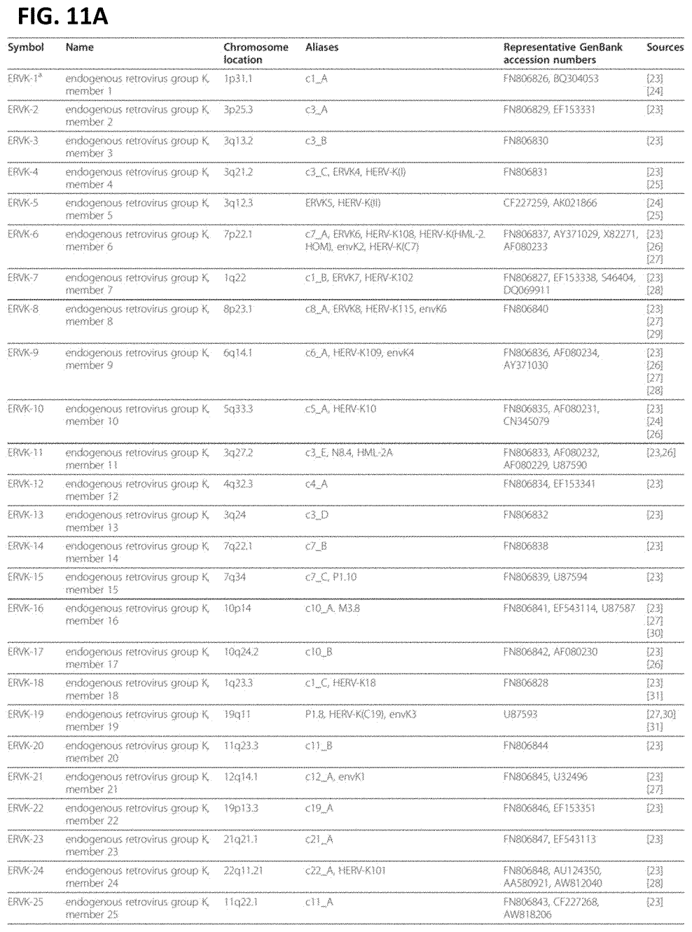

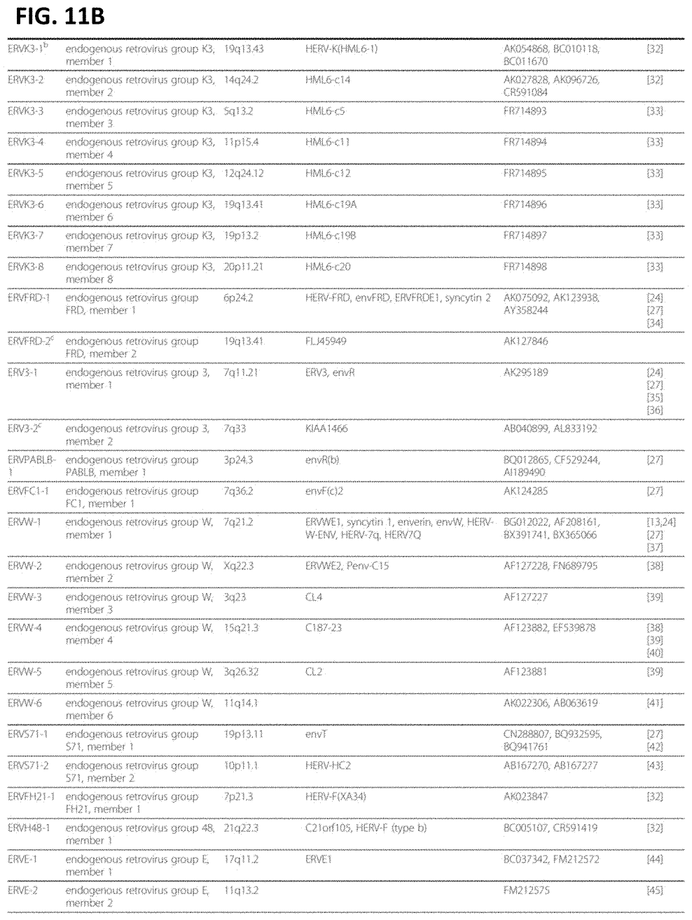

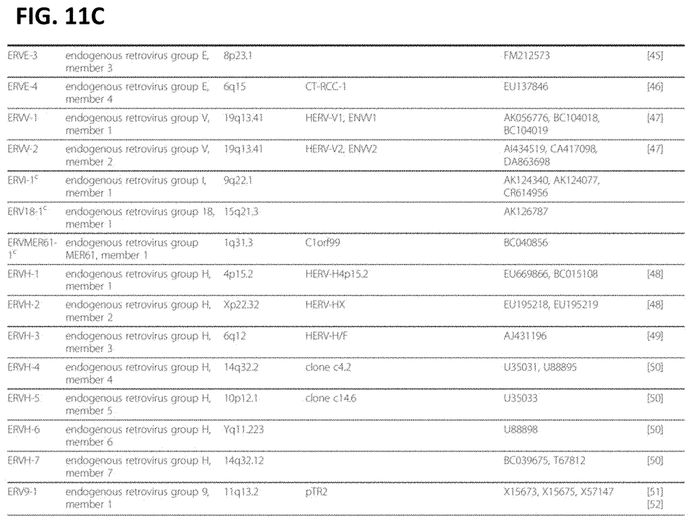

πERV-high tumors in these cancers were also enriched in tumors with known potential predictors of ICA, namely in tumors with APOBEC mutagenesis (Roberts et al., Nat Genet., 45(9):970-6, 2013) in ER+ HER2−, MSI-H tumors in COAD, and HPV+ tumors in HNSC (FIGS. 7E, 8E, and 9E). Although hyper-mutation due to APOBEC and MSI-H etiologies are known to be associated with ICA in ER+ HER2− and COAD, respectively (Panda et al., JCO Precis Oncol., 2017), a detailed analysis (FIGS. 10A-10C) demonstrated that πERV-high tumors showed evidence of immune activation and checkpoint pathway up-regulation compared to πERV-low tumors in ER+ HER2−, both with and without APOBEC mutagenesis, as well as in both MSI-H and MSI-L/MSS in COAD and in both HPV+ and HPV− in HNSC. These results demonstrate that πERV expression associates with ICA independently of APOBEC mutagenesis status in ER+ HER2−, MSI-H status in COAD, and HPV status in HNSC.

### Example 6—Transcriptomic Correlates of ERV Expression Points to Chromatin Alterations

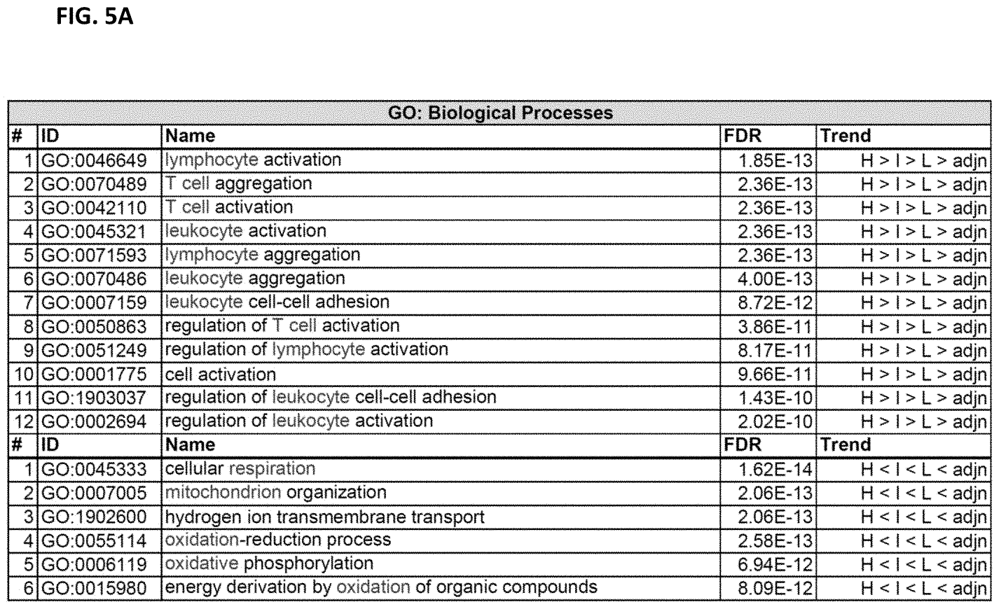

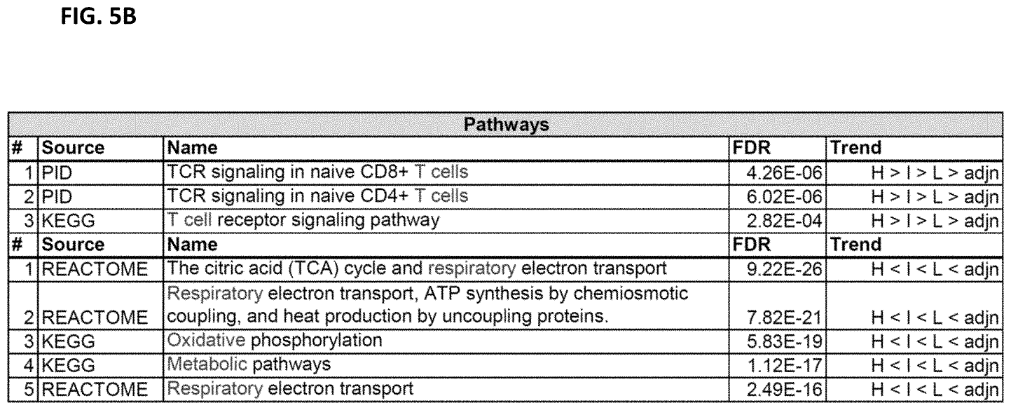

Further, analysis of the gene expression profiles of the three πERV subtypes of KIRC and adjacent normals in the TCGA dataset showed that expression levels of 1,048 genes followed the trend πERV-high>πERV-intermediate>πERV-low>adjacent normal, whereas 1,103 genes followed the trend πERV-high<πERV-intermediate<πERV-low<adjacent normal (where [symbol] means significantly higher/lower). Enrichment analysis (FIGS. 5A-5B) of these two sets of genes using the ToppGene suite (Chen et al., Nucleic Acids Res., 37(Web Server issue):W305-11, 2009) showed that the former set was enriched in immune activation genes, whereas the latter set was enriched in genes associated with mitochondrial respiration, which often plays a role in determining disease behavior.

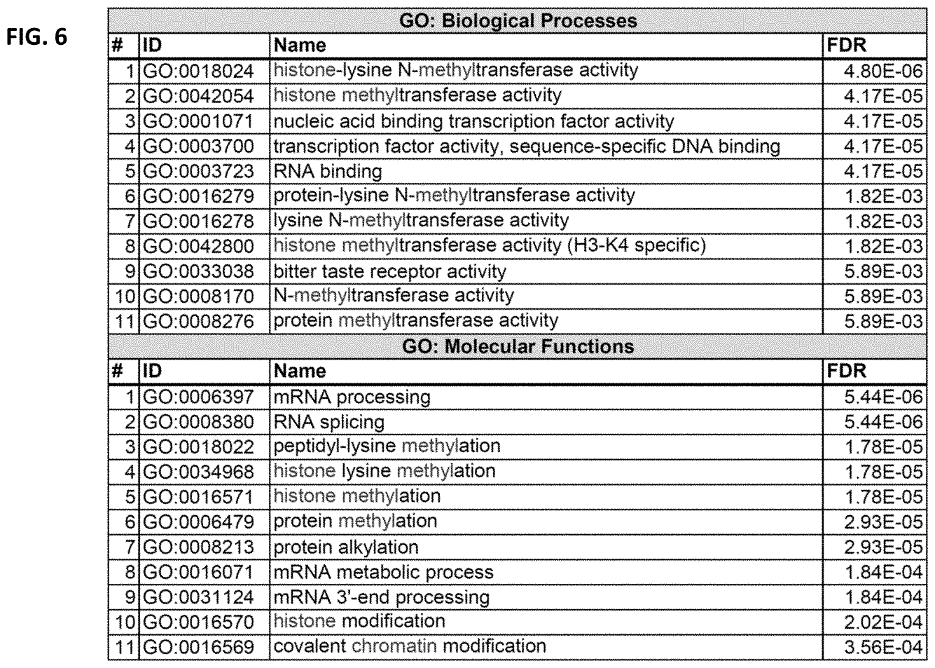

To identify the potential cause of ERV expression, genes with expression that correlated with overall ERV expression in each cancer type (details in Example 1). Six hundred fifty-seven genes satisfied this criterion in KIRC, ER+ HER2−, and COAD. Enrichment analysis of these genes using ToppGene (Chen et al., Nucleic Acids Res., 37(Web Server issue):W305-11, 2009) showed that “methyl” (methyltransferase and methylation) and “histone” pathways were significantly associated with overall ERV expression (FIG. 6). The data indicate that epigenetic alterations, specifically those involving modification of histone methylation-based control of gene expression, may be a functional mechanism of ERV expression in these cancers.

We claim:

1. A method of detecting immunogenic endogenous retroviruses (πERVs) in a subject, comprising:
measuring expression of at least one endogenous retrovirus (ERV) in a tumor sample from a subject with a tumor; and
measuring expression of at least one ERV in a control sample selected from:
a control tumor sample from a subject that is not responsive to at least one antagonist of an immune checkpoint pathway (ICP)-related molecule; and/or
a control sample from a subject without a tumor,
wherein the at least one ERV is an πERV if the ERV expression in the tumor sample from the subject is at least 1.5 greater than the control sample.

2. The method of claim 1, wherein if at least one πERV in the tumor sample is detected, further comprising administering a therapeutically effective amount of a PD-1 antagonist, PD-L1 antagonist, CTLA4 antagonist, BTLA antagonist, HVEM antagonist, LAG-3 antagonist, or combinations thereof, to the subject having the tumor, thereby treating the tumor.

3. The method of claim 1, wherein expression of the πERV is correlated with upregulation of at least one ICP.

4. The method of claim 1, wherein the tumor sample from the subject responsive to at least one antagonist of an ICP-related molecule and/or the tumor sample from the subject that is not responsive to at least one antagonist of an ICP-related molecule is a solid tumor.

5. The method of claim 4, wherein the solid tumor is a clear cell renal cell carcinoma, ER+ HER2− breast cancer, colon cancer, or head and neck squamous cell cancer.

6. The method of claim 1, wherein the at least one ERV comprises one or more of ERVK.3, ERV3-2, ERVK7, EVER24, or ERVK.8.

7. The method of claim 6, wherein the at least one ERV comprises ERV3-2.

8. The method of claim 2, wherein the PD-1 antagonist, PD-L1 antagonist, CTLA4 antagonist, BTLA antagonist, HVEM antagonist, OR LAG-3 antagonist comprises a monoclonal antibody.

9. The method of claim 8, wherein the PD-1 or PD-L1 antagonist comprises one or more of tezolizumab, MPDL3280A, BNS-936558 (Nivolumab), pembrolizumab, pidilizumab, CT011, AMP-224, AMP-514, MEDI-0680, BMS-936559, BMS935559, MEDI-4736, MPDL-3280A, MSB-0010718C, MGA-271, indoximod, epacadostat, BMS-986016, MEDI-4736, MEDI-4737, MK-4166, BMS-663513, PF-05082566 (PF-2566), lirilumab, and durvalumab.

10. The method of claim 9, wherein the CTLA4 antagonist comprises tremelimumab, ipilimumab, or both.

11. The method of claim 1, wherein measuring expression of at least one ERV comprises measuring ERV nucleic acid expression.

12. The method of claim 1, wherein measuring ERV nucleic acid expression comprises amplification of ERV nucleic acid molecules.

13. The method of claim 12, comprising measuring ERV nucleic acid expression using the ΔΔCt method.

14. The method of claim 13, comprising normalizing the ERV nucleic acid expression to nucleic acid expression of a housekeeping gene.

15. The method of claim 14, wherein the housekeeping gene comprises hypoxanthine-guanine phosphoribosyltransferase (HPRT1).

16. The method of claim 12, wherein measuring ERV nucleic acid expression comprises using:
(SEQ ID NO: 1)
(a) 5′-CAAGAGGCGGCATAGAAGCAA-3′
and
(SEQ ID NO: 2)
5′-GGAGAGTAGCTTGGGGTTTCA-3′;
(SEQ ID NO: 3)
(b) 5′-AGCCATTTACAAAGAAAGGGGAC-3′
and
(SEQ ID NO: 4)
5′-CTATGCCGCCTCTTGTCTGAT-3′;
or
(c) both (a) and (b).

17. The method of claim 15, wherein measuring HPRT1 expression comprises using:
(SEQ ID NO: 5)
5′-GACACTGGCAAAACAATGCAGAC-3′;
and
(SEQ ID NO: 6)
5′-TGGCTTATATCCAACACTTCGTGG-3′.

18. A method of treating cancer, comprising:
selecting a subject with cancer;
detecting at least one immunogenic endogenous retrovirus (πERV) in the subject, comprising:
measuring expression of at least one endogenous retrovirus (ERV) in a tumor sample from the subject, wherein at least one ERV is an πERV if the ERV expression is at least 1.5-fold greater than a control sample, wherein the control sample comprises expression of the at least one ERV expected for at least one of:
a tumor sample from a subject with cancer that is not responsive to at least one antagonist of an ICP-related molecule; and/or
a sample from a subject without a tumor; and
administering a therapeutically effective amount of a PD-1 antagonist, PD-L1 antagonist, CTLA4 antagonist, BTLA antagonist, HVEM antagonist, LAG-3 antagonist, or combinations thereof, thereby treating the cancer.

19. The method of claim 18, wherein measuring expression of at least one ERV comprises measuring ERV3-2 nucleic acid expression.

20. A method of detecting immunogenic endogenous retroviruses (πERVs), comprising:
measuring expression of at least one endogenous retrovirus (ERV) in a tumor sample from a subject responsive to at least one antagonist of an immune checkpoint pathway (ICP)-related molecule; and
measuring expression of at least one control sample from at least one of:
a tumor sample from a subject that is not responsive to at least one antagonist of an immune checkpoint pathway (ICP)-related molecule; and/or
a sample from a subject without a tumor.


SEQUENCE LISTING (8 sequences)

SEQ ID NO: 1 - DNA, 21 bp, Artificial Sequence
  primer
caagaggcggcatagaagcaa

SEQ ID NO: 2 - DNA, 21 bp, Artificial Sequence
  primer
ggagagtagcttggggtttca

SEQ ID NO: 3 - DNA, 23 bp, Artificial Sequence
  primer
agccatttacaaagaaaggggac

SEQ ID NO: 4 - DNA, 21 bp, Artificial Sequence
  primer
ctatgccgcctcttgtctgat

SEQ ID NO: 5 - DNA, 23 bp, Artificial Sequence
  primer
gacactggcaaaacaatgcagac

SEQ ID NO: 6 - DNA, 24 bp, Artificial Sequence
  primer
tggcttatatccaacacttcgtgg

SEQ ID NO: 7 - DNA, 1384 bp, Artificial Sequence
  ERV3-2
atcaacattgtctgtaattcagcttatgttgtaaacgtagccagtcacattcacatggaaactgctacagttaaaagtacactagacccagaactgcttaatttgtttcacttattctcaaatgctgcatgctacatgccaaacaggtgagacagctggtcatgtacagtgacattgtctgtcatcatttgctcatatggggatacttaaacaattaaaaactgacaatggactcgcttatactagtcatgcttttcaaaatttcttatagctttgggctataacccataaaattggaattccttataatcctaaaggacaaggcattgtagagcaggcacattgaacattacaacacatgttgaaaagacgaaaggggtataggaggccaactaccacctcaatcaaaactacatttagccttatttacttcacatttttggactcctggtacagatggtaagactccagcagaaagacattggcaagtgttagaggaaaagaggaaagtttatctgaaagcattatggaaatccctggaaggacaatggaaaggttgggtggatttactgacgtggagaagagggtatgcttgtgttttcacaggagatggacaagccgtgtgggtgccctcaaggtgtgtgcaaccatggaatgggagactggaggaacccagggtggccaaccatgggtccggtccctctggtgtgagccatgagccagctgagcctgagtgcgaagacggagagaaggccgaccacagtcatgacatcaacccccataacctggggacaactcaagaaaaccacacaggaggctgagaaactactggagcaccagggacagtctgtaaagttggatggaccaccaatgggaaaatgagagctgcccaccctggccttacactccttcaattaatacataaacagaaaggaggatatacagagagccaaaggcccatgggacgtgaccaacattccactgagtctatacgatcaaacagcaaactgtttatcatgaatacagaatgtgggcaaactcatgactgtgcctgccccagaaggtttgctgagggcaattgcttcctgacgccaagctccttgaggttatctattgggacatccagagaatgcagtcttgcaagcctactctggaccgagcagctgaccccctcttccacttcccttcacactatctcttttgcctaataaatacggaaggctgtgtacaaggtcaggtcccttgtccactagaggcaaggtgcttcctgacccttcttccaaatataaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 8 - protein, 109 aa, Artificial Sequence
  ERV3-2
MRAAHPGLTLLQLIHKQKGGYTESQRPMGRDQHSTESIRSNSKLFIMNTECGQTHDCACPRRFAEGNCFLTPSSLRLSIGTSRECSLASLLWTEQLTPSSTSLHTISFA